(19) United States
(12) Patent Application Publication
(10) Pub. No.: US 2025/0375523 A1
(43) Pub. Date: Dec. 11, 2025

(54) COMPOSITIONS AND SYSTEMS FOR COMBINATORIAL THERAPIES CONTAINING FUCOSYLATED CELLS AND IMMUNE CHECKPOINT INHIBITORS AND METHODS OF PRODUCTION AND USE THEREOF

(71) Applicant: Targazyme, Inc., Carlsbad, CA (US)

(72) Inventors: Reid P. BISSONNETTE, Carlsbad, CA (US); Lynnet KOH, Carlsbad, CA (US)

(21) Appl. No.: 19/127,912

(22) PCT Filed: Nov. 10, 2023
(86) PCT No.: PCT/US2023/037141

§ 371 Date: May 7, 2025

Related U.S. Application Data
(60) Provisional application No. 63/383,391, filed on Nov. 11, 2022.

(51) Int. Cl. A61K 40/11; A61K 38/20; A61P 35/00; C12N 5/0783

(52) U.S. Cl. CPC A61K 40/11; A61K 38/2013; A61P 35/00; C12N 5/0638; C12N 2501/724

(57) ABSTRACT

Compositions, systems, and kits are disclosed that comprise at least one immune checkpoint inhibitor and at least one immune cell type for adoptive cell therapy, wherein the at least one immune cell type has been fucosylated ex vivo (fuco-ACT). Also disclosed are methods of making and using the compositions, systems, and kits.

### CROSS REFERENCE TO RELATED APPLICATIONS/INCORPORATION BY REFERENCE STATEMENT

The subject application claims benefit under 35 USC § 119 (e) of U.S. Provisional Application No. 63/383,391, filed Nov. 11, 2022. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

Cancer is a significant cause of morbidity and mortality worldwide. While the standards of care for many different cancer types have greatly improved over the years, current standards of care still fail to meet the need for effective therapies to improve the treatment of cancer. The renaissance of cancer immunotherapies, which include antibodies, vaccines, cytokines, oncolytic viruses, bispecific molecules, and cellular therapies, is demonstrated by the approval of several immunotherapeutic antibody agents targeting, for example (but not by way of limitation), cytotoxic T-lymphocyte-associated protein 4 (CTLA-4), the programmed cell death receptor-1 (PD-1) and its ligand PD-L1, and the lymphocyte-activation gene 3 (LAG-3), collectively referred to as immune checkpoint inhibitors, (ICIs). The renaissance of cancer immunotherapies is also demonstrated by the approval of several cell-based immunotherapies, collectively referred to as adoptive cell therapies (ACTs). ACTs are generated by a process which involves the isolation of a patient's own immune cells followed by their ex vivo expansion and reinfusion. The majority of adoptive cellular therapy strategies utilize T cells isolated from tumor or peripheral blood but may utilize other immune cell subsets or cells derived from inducible pluripotent stems cells (iPSCs). T-cell therapies in the form of tumor-infiltrating lymphocytes (TILs), T-cell receptor gene-transduced T cells (TCR-T cells), and chimeric antigen receptor gene-transduced T cells (CAR-T cells) act as “living drugs” as these infused cells expand, engraft, and persist in vivo, allowing adaptability over time and enabling durable remissions in subsets of patients. Adoptive cellular therapy has been less successful in the management of solid tumors because of poor homing, proliferation, and survival of transferred cells.

### BRIEF DESCRIPTION OF THE DRAWINGS

FIG. 1 graphically depicts one non-limiting embodiment of a combinatorial therapy method constructed in accordance with the present disclosure, wherein the combinatorial therapy includes the use of immune checkpoint inhibitors with tumor infiltrating lymphocytes that have been fucosylated ex vivo.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the medical procedures and techniques of, surgery, anesthesia, wound healing, and infectious control described herein are those well-known and commonly used in the art. Standard techniques are used for infection diagnostic and therapeutic applications.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, systems, kits, and/or methods disclosed herein can be made and executed without undue experimentation in light of the present disclosure. While the articles, systems, kits, and/or methods have been described in terms of particular embodiments, it will be apparent to those of skill in the art that variations may be applied to the articles, systems, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example (but not by way of limitation), when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with,” “coupled to,” and “connected to” include both direct association/coupling/connection of two elements to one another as well as indirect association/coupling/connection of two elements to one another. When two elements are indirectly associated/coupled/connected to one another, one or more intervening elements may be present therebetween (such as, but not limited to, a linking moiety).

All patents, applications, published applications, and other publications cited herein are incorporated by reference in their entirety. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices, and materials similar or equivalent to those described herein can be used in the practice of the present disclosure. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the present disclosure.

The term “adoptive cell therapy” or “ACT” refers to the transfer of cells into a patient. The cells may have originated from the patient, another individual, or an inducible pluripotent stem cell (iPSC).

The term “tumor-infiltrating lymphocyte” or “TIL” refers to all lymphocytic cell populations that are capable of invading tumor tissue. TIL therapy is a form of ACT that involves harvesting infiltrated lymphocytes from tumors or circulating lymphocytes, culturing and amplifying them in vitro, and then infusing the cultured/amplified cells into one or more patients for treatment of a condition or disorder.

The term “chimeric antigen receptor T-cell” or “CAR-T cell” refers to T cells that have been genetically engineered to produce an artificial T cell receptor for use in immunotherapy. CAR-T cell therapy is a form of ACT that involves harvesting circulating lymphocytes from the patient, a separate donor, or iPSC; genetically engineering the harvested lymphocytes; culturing and amplifying the genetically engineered cells in vitro; and infusing the cultured/amplified genetically engineered lymphocytes into one or more patients for treatment of a condition or disorder.

The term “chimeric antigen receptor NK cell” or “CAR-NK cell” refers to NK cells that have been genetically engineered to produce an artificial T cell receptor for use in immunotherapy. CAR-NK cell therapy is a form of ACT that involves harvesting circulating NK cells from the patient, separate donor, or iPSC; genetically engineering the NK cells; culturing and amplifying the genetically engineered NK cells in vitro; and then infusing the cultured/amplified genetically engineered NK cells into one or more patients for treatment of a condition or disorder.

The term “PD-1 inhibitor” refers to a moiety (e.g., compound, nucleic acid, polypeptide, antibody or fragment thereof, etc.) that decreases, inhibits, blocks, abrogates, or interferes with the activity or expression of PD-1 (e.g., Programmed Cell Death Protein 1; PD-1 (CD279); GI: 145559515), including variants, isoforms, species homologs of human PD-1 (e.g., mouse) and analogs that have at least one common epitope with PD-1. A PD-1 inhibitor includes molecules and macromolecules such as (but not limited to) compounds, nucleic acids, polypeptides, antibodies, peptibodies, diabodies, minibodies, single-chain variable fragments (ScFv), and fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC₅₀). PD-1 inhibitors include exemplary compounds and compositions described herein. A PD-1 antibody refers to a PD-1 inhibitor which is a monoclonal or polyclonal antibody as described herein.

The term “PD-L1 inhibitor” refers to a moiety (e.g., compound, nucleic acid, polypeptide, antibody or fragment thereof, etc.) that decreases, inhibits, blocks, abrogates, or interferes with the activity, binding of PD-L1 to its receptor, PD-1, or expression of PD-L1 (e.g., Programmed Cell Death 1 Ligand; PD-L1 (CD274); GI: 30088843), including variants, isoforms, species homologs of human PD-L1 (e.g., mouse) and analogs that have at least one common epitope with PD-L1. A PD-L1 inhibitor includes molecules and macromolecules such as (but not limited to) compounds (small molecule compounds), nucleic acids, polypeptides, antibodies, peptibodies, diabodies, minibodies, single-chain variable fragments (ScFv), and fragments or variants thereof. Thus, a PD-L1 inhibitor as used herein refers to any moiety that antagonizes PD-L1 activity, its binding to PD-1, or its expression. PD-L1 inhibitor efficacy can be measured, for example, by its inhibitor concentration at 50% (half-maximal inhibitor concentration or IC₅₀). PD-L1 inhibitors include exemplary compounds and compositions described herein. A PD-L1 inhibitor antibody refers to a PD-L1 inhibitor which is a monoclonal or polyclonal antibody as described herein.

The term “LAG-3 inhibitor” refers to a moiety (e.g., compound, nucleic acid, polypeptide, antibody or fragment thereof, etc.) that decreases, inhibits, blocks, abrogates, or interferes with the activity or expression of LAG-3 (e.g., Lymphocyte Activation Gene 3; LAG-3 (CD223); GI: 251757512), including variants, isoforms, species homologs of human LAG-3 (e.g., mouse), and analogs that have at least one common epitope with PD-1. A LAG-3 inhibitor includes molecules and macromolecules such as (but not limited to) compounds, nucleic acids, polypeptides, antibodies, peptibodies, diabodies, minibodies, single-chain variable fragments (ScFv), and fragments or variants thereof. Thus, a LAG-3 inhibitor as used herein refers to any moiety that antagonizes LAG-3 activity or expression. LAG-3 inhibitor efficacy can be measured, for example, by its inhibitor concentration at 50% (half-maximal inhibitor concentration or IC₅₀). LAG-3 inhibitors include exemplary compounds and compositions described herein. A LAG-3 antibody refers to a LAG-3 inhibitor which is a monoclonal or polyclonal antibody as described herein.

The terms “polypeptide” and “protein” are used interchangeably herein and refer to any molecule that includes at least two or more amino acids.

The term “effective amount” refers to the amount of a therapy (e.g., each active agent, a combination of agents, or another active agent such as an anti-cancer agent described herein) which is sufficient to accomplish a stated purpose or otherwise achieve the effect for which it is administered. An effective amount can be sufficient to reduce and/or ameliorate the progression, development, recurrence, severity, and/or duration of a given disease, disorder, or condition and/or a symptom related thereto, or can be sufficient to reduce the level of activity or binding of a polypeptide (e.g., PD-1, PD-L1, or LAG-3). An effective amount can be a “therapeutically effective amount” which refers to an amount sufficient to provide a therapeutic benefit, such as (but not limited to) the reduction or amelioration of the advancement or progression of a given disease, disorder, or condition, reduction or amelioration of the recurrence, development or onset of a given disease, disorder, or condition, and/or to improve or enhance the prophylactic or therapeutic effect(s) of another therapy (ies). A therapeutically effective amount of one or more composition(s) described herein can enhance the therapeutic efficacy of another therapeutic agent.

The term “regimen” refers to a protocol for dosing and timing the administration of one or more therapies (e.g., combinations described herein or another active agent such as an anti-cancer agent described herein) for treating a disease, disorder, or condition described herein. A regimen can include periods of active administration and periods of rest as known in the art. Active administration periods include administration of compositions and combinations described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the compositions and combinations described herein.

The terms “therapy” and “therapies” as used herein refer to any protocol(s), method(s), and/or agent(s) that can be used in the prevention, treatment, management, and/or amelioration of a disease, disorder, or condition or one or more symptoms thereof. In certain non-limiting instances, the term(s) refers to the administration of one or more active agents to a patient. In other non-limiting instances, the term(s) refers to one or more procedures performed on a patient.

The term “patient” or “subject” refers to a mammal, such as a human, bovine, rat, mouse, dog, monkey, ape, goat, sheep, cow, or deer. Generally, a patient as described herein is human.

The terms “inhibition,” “inhibit,” and “inhibiting” refer to a reduction in the activity, binding, or expression of a polypeptide or to a reduction or amelioration of a disease, disorder, or condition or a symptom thereof. “Inhibiting” as used herein can include partially or totally blocking stimulation, decreasing, preventing, or delaying activation or binding, or inactivating, desensitizing, or down-regulating protein or enzyme expression, activity, or binding.

Antibodies described herein can be polyclonal or monoclonal and include xenogeneic, allogeneic, or syngeneic forms and modified versions thereof (e.g., humanized or chimeric). An “antibody” is intended to mean a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See Borrebaeck (ed.) (1995) Antibody Engineering, Second Edition, Oxford University Press.; Kuby (1997) Immunology, Third Edition, W.H. Freeman and Company, New York). Specific molecular antigens that can be bound by an antibody described herein include PD-1, PD-L1, LAG-3, etc. and any epitopes thereof.

The term “monoclonal antibody (ies)” refers to a population of antibody molecules that contain one species of an antigen binding site capable of immunoreacting with a particular epitope of an antigen, whereas the term “polyclonal antibody (ies)” refers to a population of antibody molecules that contain multiple species of antigen binding sites capable of interacting with a particular antigen. A monoclonal antibody typically displays a single binding affinity for a particular antigen with which it immunoreacts. For example, the monoclonal antibodies to be used in accordance with the present disclosure can be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)); recombinant DNA methods (see, e.g., U.S. Pat. No. 4,816,567); phage-display technologies (see, e.g., Clackson et al., Nature, 352:624-628 (1991); Marks et al., J Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004)); and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lon berg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995)).

The monoclonal antibodies herein also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, pp. 6851-6855 (1984)). “Humanized antibody (ies)” can be considered as a subset of chimeric antibodies described herein.

The term “human” when used in reference to an antibody or a functional fragment thereof (e.g., “humanized antibody (ies))” refers to an antibody or functional fragment thereof that has a human variable region or a portion thereof corresponding to human germline immunoglobulin sequences. Such human germline immunoglobulin sequences are described by Kabat et al. (Sequences of Proteins of Immunological Interest, Fifth Edition, U.S. Department of Health and Human Services, NIH Publication No. 91-3242 (1991)). A human antibody, in the context of the present disclosure, can include an antibody that binds to PD-1, PD-L1, LAG-3, etc. or variants thereof as described herein.

In certain instances, a human antibody is an antibody that possesses an amino acid sequence corresponding to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boemer et al., J. Immunol., 147 (1): 86-95 (1991). See also van Dijk and van de Winkel, Curr. Opin. Pharmacol., 2:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE technology). See also, for example, Li et al., Proc. Natl. Acad. Sci. USA, 103:3557-3562 (2006) regarding human antibodies generated via a human B-cell hybridoma technology.

A “humanized antibody” refers to antibodies made by a non-human cell having variable or variable and constant regions which have been altered to more closely resemble antibodies that would be made by a human cell. For example, by altering the non-human antibody amino acid sequence to incorporate amino acids found in human germline immunoglobulin sequences. The humanized antibodies of the present disclosure can include amino acid residues not encoded by human germline immunoglobulin sequences (e.g., mutations introduced by random or site-specific mutagenesis in vitro or by somatic mutation in vivo), for example in the CDRs. Humanized antibodies can also include antibodies in which CDR sequences derived from the germline of another mammalian species, such as a mouse, have been grafted onto human framework sequences.

Humanized forms of non-human (e.g., murine) antibodies are antibodies that contain minimal sequence derived from non-human immunoglobulin. In one embodiment, a humanized antibody is a human immunoglobulin (recipient antibody) in which residues from a hypervariable region of the recipient are replaced by residues from an hypervariable region of a nonhuman species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions can include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally can also include at least a portion of an immunoglobulin constant region (Fc), which can be a human immunoglobulin. Exemplary methods and humanized antibodies include those described by Jones et al. Nature 321:522-525 (1986); Riechmann et al. Nature 332:323-329 (1988); and Presta, Curr. Op. Struct. Biol. 2:593-596 (1992); Vaswani and Hamilton, Ann. Allergy. Asthma & Immunol. 1:105-115 (1998); Harris, Biochem. Soc. Transactions 23:1035-1038 (1995); Burle and Gross, Curr. Op. Biotech. 5:428-433 (1994); and U.S. Pat. Nos. 6,982,321 and 7,087,409.

The term “functional fragment” when used in reference to an antibody refers to a portion of the antibody including heavy or light chain polypeptides that retains some or all of the binding activity as the antibody from which the fragment was derived. Such functional fragments can include, for example, an Fd, Fv, Fab, F(ab′), F(ab) 2, F(ab′) 2, single chain Fv(ScFv), diabody, triabody, tetrabody, and minibody. Other functional fragments can include, for example, heavy or light chain polypeptides, variable region polypeptides or CDR polypeptides or portions thereof so long as such functional fragments retain binding activity. Such antibody binding fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990). Antibody Engineering, Second Edition, Oxford University Press, 1995.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (a), delta (8), epsilon (E), gamma (γ) and mu (u), based on the amino acid sequence of the heavy chain constant region. The distinct heavy chains differ in size: α, δ, and γ contain approximately 450 amino acids, while u and & contain approximately 550 amino acids. When combined with a light chain, these distinct types of heavy chains give rise to five well known classes of antibodies, IgA, IgD, IgE, IgG, and IgM, respectively, including four subclasses of IgG, namely lgG1, lgG2, lgG3, and lgG4. A heavy chain can be (for example, but not by way of limitation) a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (A) based on the amino acid sequence of the constant domains. Light chain amino acid sequences are well known in the art. A light chain can be (for example, but not by way of limitation) a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains can differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5th Ed. A variable region can be (for example, but not by way of limitation) a human variable region.

A CDR refers to one of three hypervariable regions (H1, H2, or H3) within the non-framework region of the immunoglobulin (Ig or antibody) VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

The term “cancer” refers to any physiological condition in mammals characterized by unregulated cell growth. Cancers described herein include solid tumors and hematological (blood) cancers. A “hematological cancer” refers to any blood borne cancer and includes, for example (but not by way of limitation), myelomas, lymphomas, leukemias, and the like. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering at least one composition or combination described herein into a subject by such routes as (for example, but not by way of limitation) oral, mucosal, topical, suppository, intravenous, parenteral, intraperitoneal, intramuscular, intralesional, intrathecal, intranasal, or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more agents (e.g., the two active agents described herein and/or the two active agents plus another active agent such as (but not limited to) an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The composition(s) of the present disclosure can be administered alone or can be coadministered to the patient. Coadministration is meant to include simultaneous or sequential administration of the compound individually or in combination. Thus, the preparations can also be combined, when desired, with other active substances (e.g., to reduce metabolic degradation). The compounds described herein can be used in combination with one another or with other active agents known to be useful in treating cancer.

The term “anti-cancer agent” is used in accordance with its plain ordinary meaning and refers to a composition having anti-neoplastic properties or the ability to inhibit the growth or proliferation of cells. In certain non-limiting embodiments, an anti-cancer agent is a chemotherapeutic. In certain non-limiting embodiments, an anti-cancer agent is an agent identified herein having utility in methods of treating cancer. In certain non-limiting embodiments, an anti-cancer agent is an agent approved by the FDA or similar regulatory agency of a country other than the USA, for treating cancer.

As used herein, “Current Good Manufacturing Practice” or “cGMP” refers to the Current Good Manufacturing Practice regulations enforced by the US Food and Drug Administration (FDA) or equivalent regulatory authorities in non-US countries. cGMP regulations provide for systems that assure proper design, monitoring, and control of manufacturing processes and facilities. Adherence to the cGMP regulations assures the identity, strength, quality, and purity of drug products by requiring that manufacturers of medications adequately control manufacturing operations. This includes establishing strong quality management systems, obtaining appropriate quality raw materials, establishing robust operating procedures, detecting and investigating product quality deviations, and maintaining reliable testing laboratories.

As used herein, the term “ex vivo expansion” or “expansion” refers to a method of growing a cell population in tissue culture that increases the number of cells in that population. Cells that have undergone ex vivo expansion are referred to as “expanded”.

As used herein, the term “fucosylation” refers to the treatment of a population of cells with an a1,3-fucosyltransferase and fucose donor under conditions that increase the ability of the cells to bind to a selectin or that increase the reactivity of the cells with an antibody known in the art to bind to sLeX including, but not limited to, the HECA-452 monoclonal antibody. Cells that have been treated with an a1,3-fucosyltransferase and fucose donor and then exhibit increased binding to selectins or to the HECA-452 monoclonal antibody or to another antibody specific for sLeX are referred to as being “fucosylated.” As used herein, “fucosylation” can also refer to the levels of sLeX present on a cell population.

Turning now to the various inventive concepts, the present disclosure relates to a combinatorial therapy that includes at least one immune checkpoint inhibitor with an adoptive cell immunotherapy, wherein isolated immune cells (and/or genetically modified versions thereof) are enhanced by cell surface modification using fucosyltransferase enzymes that add fucose to the immune cells and upregulate selectin ligands on the immune cells. The combinatorial therapy is useful for treating cancer, including (but not limited to) reducing and/or preventing cancer metastasis. The combination is also useful for treating cancers, including those that have been previously treated with any of 1) an immune checkpoint inhibitor and/or 2) an adoptive cell immunotherapy.

Certain non-limiting embodiments of the present disclosure are related to a composition (such as, but not limited to, a pharmaceutical composition) that includes at least one immune checkpoint inhibitor (ICI) and at least one isolated immune cell type that has been fucosylated ex vivo (fuco-ACT). The composition may include one or more additional agents; for example, when the composition is a pharmaceutical composition, the composition may further include at least one pharmaceutically acceptable carrier. In addition, the composition may include one or more additional active agents, as described in further detail herein below.

Certain non-limiting embodiments of the present disclosure are related to a system that includes a composition comprising at least one immune checkpoint inhibitor (ICI) and a composition comprising at least one isolated immune cell type that has been fucosylated ex vivo (fuco-ACT). The system (and the compositions present therein) may further include one or more additional agents, as described in further detail herein below.

Any immune checkpoint inhibitors known in the art or otherwise contemplated herein may be utilized in accordance with the present disclosure. Non-limiting examples of immune checkpoint inhibitors that can be utilized include PD-1 inhibitors, PD-L1 inhibitors, LAG-3 inhibitors, CTLA-4 inhibitors, TIM-3 inhibitors, B7-H3 inhibitors, A2aR inhibitors, CD73 inhibitors, NKG2A inhibitors, PVRIG/PVRL2 inhibitors, CEACAM1 inhibitors, FAK inhibitors, CCL2/CCR2 inhibitors, LIF inhibitors, CD47/SIRPa inhibitors, CSF-1 inhibitors, IL-1 inhibitors, IL-8 inhibitors, SEMA4D inhibitors, Ang-2 inhibitors, CLEVER-1 inhibitors, phosphatylylserine inhibitors, and the like, as well as any combinations thereof.

The immune checkpoint inhibitors may be any molecule (or combinations of molecules) capable of inhibiting, blocking, abrogating or interfering with the activity or expression of any of the proteins disclosed herein above. For example (but not by way of limitation), the immune checkpoint inhibitor(s) can be a small molecule compound, a nucleic acid, a polypeptide, an antibody, a peptibody, a diabody, a minibody, a single-chain variable fragment (ScFv), or a functional fragment or variant thereof. In one instance, the immune checkpoint inhibitor is a small molecule compound (e.g., a compound having a molecule weight of less than about 1000 Da.) In other instances, useful immune checkpoint inhibitors include nucleic acids and polypeptides. In addition, the immune checkpoint inhibitor can be a polypeptide (e.g., macrocyclic polypeptide) such as those exemplified in U.S. Patent Application Publication No. US 2014/0294898.

In other alternatives, the immune checkpoint inhibitor is an antibody (i.e., a monoclonal or polyclonal antibody) or a functional fragment thereof, such as but not limited to, human antibodies, mouse antibodies, chimeric antibodies, humanized antibodies, or chimeric humanized antibodies. In one non-limiting embodiment, the immune checkpoint inhibitor is a human antibody. In another non-limiting embodiment, the immune checkpoint inhibitor is a mouse antibody. In still another non-limiting embodiment, the immune checkpoint inhibitor is a chimeric antibody. In yet another non-limiting embodiment, the immune checkpoint inhibitor is a humanized antibody. In yet another non-limiting embodiment, the immune checkpoint inhibitor is a chimeric humanized antibody.

The specificity of a PD-1 or PD-L1 or LAG-3 antibody or functional fragment thereof refers to the ability of an individual antibody or functional fragment thereof to react with only one antigen (e.g., a single epitope of PD-1 or PD-L1 or LAG-3). An antibody or functional fragment can be considered specific when it can distinguish differences in the primary, secondary or tertiary structure of an antigen or isomeric forms of an antigen.

Non-limiting examples of PD-1 inhibitors that can be utilized in accordance with the present disclosure include nivolumab (OPDIVO®, Bristol Meyers Squibb); pembrolizumab (KEYTRUDA®, Merck & Co.); cemiplimab (LIBTAYO®, Regeneron Pharmaceuticals); pidilizumab (Medivation); dostarlimab (JEMPERLI®, Glaxosmithkline); pimivalimab (Jounce Therapeutics); spartalizumab (Novartis); camrelizumab (AiRuiKa™, Jiangsu Hengrui Medicine); sintilimab (TYVYT®, Eli Lilly); tislelizumab (BeiGene); toripalimab (Tuoyi™, Shanghai Junshi Bioscience); retifanlimab (Incyte); and the like, as well as combinations thereof.

Non-limiting examples of PD-L1 inhibitors that can be utilized in accordance with the present disclosure include atezolizumab (TECENTRIQ®, Genentech); durvalumab (IMFINZI®, Medimmune/AstraZeneca); avelumab (BAVENCIDO, Pfizer); cosibelimab (Checkpoint Therapeutics); envafolimab (TRACON Pharmaceuticals); AUNP12 (Aurigene); socazolimab (Lee's Pharmaceutical/Sorrento Therapeutics); STI-3031 (Sorrento Therapeutics); and the like, as well as combinations thereof.

Non-limiting examples of LAG-3 inhibitors that can be utilized in accordance with the present disclosure include ieramilimab (LAG525, Novartis); Sym022 (Symphogen); TSR-033 (GlaxoSmithKline); fianlimab (Regeneron); leramilimab (Novartis); INCAGN2385-101 (Incyte Biosciences); favezelimab (Merck & Co.); BI754111 (Boehringer Ingelheim); and the like, as well as combinations thereof.

Other non-limiting examples of immune checkpoint inhibitors that may be utilized in accordance with the present disclosure include a CTLA-4 inhibitor (Ipilimumab (Yervoy), a TIM-3 inhibitor (MBG453); a B7-H3 inhibitor (MGC018); an A2aR inhibitor (EOS100850); a CD73 inhibitor (CPI-006); an NKG2A inhibitor (Monalizumab); a PVRIG/PVRL2 inhibitor (COM701); a CEACAM1 inhibitor (CM24); an FAK inhibitor (Defactinib); a CCL2/CCR2 inhibitor (PF-04136309); an LIF inhibitor (MSC-1); a CD47/SIRPa inhibitor (ALX148); a CSF-1 inhibitor (Lacnotuzumab); an IL-1 inhibitor (Canakinumab); an IL-8 inhibitor (BMS-986253); an SEMA4D inhibitor (Pepinemab); an Ang-2 inhibitor (Trebananib); a CLEVER-1 inhibitor (Enapotamab); a Phosphatylylserine inhibitor (Bavituximab); and combinations thereof.

The present disclosure may also utilize a combination of two or more immune checkpoint inhibitors. For example (but not by way of limitation), relatlimab (OPDUALAG™, Bristol Meyers Squibb) includes a PD-1 inhibitor combined with a LAG-3 inhibitor.

Any immune cells that are capable of being fucosylated may be utilized in accordance with the present disclosure. In certain particular (but non-limiting) embodiments, the immune cells include tumor-infiltrating lymphocytes (TILs). Non-limiting examples of immune cell types that can be utilized in accordance with the present disclosure include cytotoxic T cells (CTL), regulatory T cells (Treg), helper T cells, NK cells, B cells, dendritic cells, and the like, as well as combinations thereof. The immune cells may be isolated from the patient (autologous), a related or unrelated donor (allogeneic), or inducible pluripotent stem cells (iPSCs); alternatively, the immune cells may be genetically modified. Non-limiting examples of genetically modified immune cells that may be utilized in accordance with the present disclosure include chimeric antigen receptor gene-transduced T cells (CAR-T cells), T cell receptor gene-transduced T cells (TCR-T cells), chimeric antigen receptor gene-transduced natural killer (NK) cells (CAR-NK cells), and the like. In yet another non-limiting embodiment, the adoptive cell immunotherapy is derived from T cells obtained from the patient, a donor, or an iPSC, which are stimulated in the laboratory using tumor cells or products of tumor cells to produce tumor-selective immune cells.

The immune cells are fucosylated ex vivo using fucosyltransferase enzymes that add fucose to the immune cells and upregulate selectin ligands on the immune cells. Non-limiting examples of methods of fucosylating cells ex vivo that can be utilized in accordance with the present disclosure are disclosed in U.S. Pat. Nos. 7,332,334; 7,776,591; 8,084,255; 8,633,021; 9,511,095; and 10,799,538; and US Patent Application Publication Nos. US 2011/0091434; US 2014/0161782; US 2017/0121673; US 2019/0017023; US 2019/0062694; US 2023/0014609; and the like.

In certain non-limiting embodiments, the methods of fucosylating cells ex vivo comprises one or more of the following steps: 1) identifying a cancer patient in need of adoptive cell therapy; 2) isolating immune cells from the patient's blood or cancer tumor; 3) expanding the immune cells ex vivo; and 4) contacting the expanded immune cells with a fucosyltransferase and at least one of fucose or GDP-fucose to provide fucosylated immune cells.

In certain particular (but non-limiting) embodiments, the method of fucosylating cells further comprises after step (4), the step of (4a) separating fucosylated lymphocytes from other components after step (4), and wherein step (5) is further defined as returning the fucosylated lymphocytes to the patient intravenously. In a particular (but non-limiting) embodiment, the method also further comprises, after step (4a), the step of (4b) isolating specific fucosylated lymphocyte types from other fucosylated lymphocytes, and wherein step (5) is further defined as returning the specific fucosylated lymphocyte types to the patient intravenously.

Any fucosyltransferases known in the art or otherwise contemplated herein may be utilized in accordance with the present disclosure. Non-limiting examples of fucosyltransferases that may be utilized in accordance with the present disclosure include an α1,3-fucosyltransferase I (FUT1), an α1,3-fucosyltransferase II (FUT2), an α1,3-fucosyltransferase III (FUT3), an α1,3-fucosyltransferase IV (FUT4), an α1,3-fucosyltransferase V (FUT5), an α1,3-fucosyltransferase VI (FUT6), an α1,3-fucosyltransferase VII (FUT7), an α1,3-fucosyltransferase VIII (FUT8), an α1,3-fucosyltransferase IX (FUT9), an α1,3-fucosyltransferase X (FUT10), and an @1,3-fucosyltransferase XI (FUT11), or any combination thereof. Particular (but non-limiting) examples of fucosyltransferases that can be utilized in accordance with the present disclosure include FUT3, FUT5, FUT6, FUT7, FUT9, FUT10, and combinations thereof. On particular (but non-limiting) example of a fucosyltransferase that can be utilized is FUT7 (product name TZ 102, Targazyme Inc., Carlsbad, CA).

Any fucose donors known in the art or otherwise contemplated herein may be utilized in accordance with the present disclosure. Non-limiting examples thereof include fucose and GDP-fucose.

The compositions and systems of the present disclosure may include any combination of immune checkpoint inhibitor(s) and fuco-ACT(s) disclosed or otherwise contemplated herein. Particular (but non-limiting) examples of such combinations include a fuco-ACT combined with a PD-1 inhibitor (such as, but not limited to, a PD-1 antibody); a fuco-ACT combined with a PD-L1 inhibitor (such as, but not limited to, a PD-L1 antibody); a fuco-ACT combined with a LAG-3 inhibitor (such as, but not limited to, a LAG-3 antibody); and the like. In another non-limiting embodiment, the PD-1 or PD-L1 or LAG-3 inhibitor is a small molecule compound, a nucleic acid, a peptide, a protein, an antibody, a peptibody, a diabody, a minibody, a single-chain variable fragment (ScFv), or a fragment or variant thereof.

The composition(s) of the present disclosure may be provided with any formulation known in the art or otherwise contemplated herein. In certain particular (but non-limiting) embodiments, the compositions contain one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; nanocarriers; scaffolds that allowed delayed drug release (such as, but not limited to, hydrogels); buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed. (2020).

The immune checkpoint inhibitor (such as, but not limited to, PD-1 or PD-L1 or LAG-3 antibody) can be present in an amount as a measure with regards to the weight of the patient in need thereof. For example (but not by way of limitation), the immune checkpoint inhibitor can be present in an amount of about: 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The PD-1 or PD-L1 or LAG-3 antibody can be present in an amount of about: 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The immune checkpoint inhibitor can be present in an amount of about 0.5 mg/kg to about 5 mg/kg or about 0.1 mg/kg to about 10 mg/kg. The immune checkpoint inhibitor can be present in an amount of about 0.5 mg/kg to about 15 mg/kg or about 0.1 mg/kg to about 20 mg/kg.

In still other non-limiting embodiments, the immune checkpoint inhibitor can be present at an amount of about: 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg or 30 mg/kg. The PD-1 or PD-L1 or LAG-3 antibody can be present at an amount of about: 1 mg/kg, 2 mg/kg, 3 mg/kg, or 5 mg/kg.

The immune checkpoint inhibitor can be present in the combination at any amount, such as (but not limited to) about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The immune checkpoint inhibitor can be present in the combination at an amount such as (but not limited to) about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 mg to about 2000 mg.

The immune checkpoint inhibitor can be present in the combination in any amount such as (but not limited to) about: 0.1 mg/mL, 0.5 mg/mL, 1 mg/mL, 2 mg/ml, 3 mg/ml, 4 mg/mL, 5 mg/ml, 6 mg/mL, 7 mg/mL, 8 mg/ml, 9 mg/mL, 10 mg/ml, 15 mg/ml, 20 mg/mL, 25 mg/mL, 30 mg/mL, 40 mg/ml, 50 mg/ml, 60 mg/ml, 70 mg/ml, 80 mg/ml, 90 mg/ml, 100 mg/mL, 150 mg/mL, 200 mg/mL, 250 mg/mL, 300 mg/mL, 400 mg/mL, or 500 mg/ml. In one non-limiting embodiment, the immune checkpoint inhibitor is present in the combination in an amount of about: 1 mg/ml to about 10 mg/ml, 5 mg/mL to about 10 mg/mL, 5 mg/mL to about 15 mg/ml, 10 mg/ml to about 25 mg/ml; 20 mg/ml to about 30 mg/ml; 25 mg/mL to about 50 mg/ml, or 50 mg/ml to about 100 mg/mL.

In certain non-limiting instances, the therapeutically effective amount of immune checkpoint inhibitor is determined as an amount provided in a package insert provided with the immune checkpoint inhibitor. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

The fuco-ACT may be present in the compositions (and administered to the patient) in any amount that allows the fuco-ACT to function as described herein. Non-limiting examples of amounts that fall within the scope of the present disclosure include at least about 10⁶, about 10⁷, about 10⁸, about 10⁹, about 10¹⁰, about 10¹¹, about 10¹², about 10¹³, about 10¹⁴, and the like, as well as a range that falls between two of the above values.

Certain non-limiting embodiments of the present disclosure include kits that include any of the compositions and/or systems disclosed or otherwise contemplated herein.

Certain non-limiting embodiments of the present disclosure are directed to an adoptive cell therapy method that comprises the steps of: 1) identifying a cancer patient in need of adoptive cell therapy; 2) isolating immune cells from the patient's blood or cancer tumor (wherein the immune cells are any of the immune cells disclosed or otherwise contemplated herein); 3) expanding the immune cells ex vivo; 4) contacting the expanded immune cells with at least one of any of the fucosyltransferases and at least one of any of the fucose donors disclosed or otherwise contemplated herein to provide fucosylated immune cells; 5) returning the fucosylated immune cells to the patient intravenously; and 6) administering at least one of any of the immune checkpoint inhibitors disclosed or otherwise contemplated herein to the patient simultaneously or wholly or partially sequentially with the fucosylated immune cells.

In certain particular (but non-limiting) embodiments, the method further comprises, after step (4), the step of (4a) separating fucosylated lymphocytes from other components after step (4), and wherein step (5) is further defined as returning the fucosylated lymphocytes to the patient intravenously. In another particular (but non-limiting) embodiment, the method further comprises, after step (4a), the step of (4b) isolating specific fucosylated lymphocyte types from other fucosylated lymphocytes, and wherein step (5) is further defined as returning the specific fucosylated lymphocyte types to the patient intravenously.

Any of the method steps can be repeated one or more times. For example (but not by way of limitation), at least step (5) can be repeated one or more times, and/or at least step (6) is repeated one or more times.

The at least one immune checkpoint inhibitor may be administered prior to, concurrently with, and/or following the at least one fucosylated immune cell type for adoptive cell therapy (fuco-ACT).

Certain non-limiting embodiments of the present disclosure are directed to a method of treating one or more cancers and/or reducing the occurrence or severity of metastasis in a cancer patient. The method comprises the step of administering to the cancer patient, either simultaneously or wholly or partially sequentially, at least one of any of the ICIs disclosed or otherwise contemplated herein and at least one of any of the fuco-ACTs disclosed or otherwise contemplated herein. In certain particular (but non-limiting) embodiments, the ICI(s) and fuco-ACT(s) are present in the same composition; alternatively, the ICI(s) and fuco-ACT(s) are present in separate compositions that are administered simultaneously or wholly or partially sequentially (with either composition being administered first). In addition, each of the compositions may be administered to the patient one or more times.

The methods of the present disclosure may be utilized to treat any cancers, including (but not limited to) prostate cancer; skin cancer; ovarian cancer; breast cancer (such as, but not limited to, triple negative breast cancer); cancers of non-lymphoid parenchymal organs including the heart, placenta, skeletal muscle, and lung; cancers of the head and neck including various lymphomas (such as, but not limited to, mantle cell lymphoma, Non-Hodgkin B cell lymphoma, PTCL, adenoma, squamous cell carcinoma, laryngeal carcinoma, salivary carcinoma, thymomas and thymic carcinoma); leukemia; cancers of the retina; cancers of the esophagus; multiple myeloma; melanoma; colorectal cancer; lung cancer; cervical cancer; endometrium carcinoma; gallbladder cancer; liver cancer; thyroid follicular cancer; gastric cancer; non-small cell lung carcinoma; glioma; urothelial cancer; bladder cancer; prostate cancer; renal cell cancer; infiltrating ductal carcinoma; and glioblastoma multiform; and combinations thereof.

The methods of the present disclosure may optionally include one or more additional steps. For example (but not by way of limitation), the methods may include the step of administering at least one additional treatment to the primary tumor of the cancer patient, wherein the at least one additional treatment is selected from the group consisting of radiation, surgery, chemotherapy, immunotherapy, targeted therapy, hormone therapy, stem cell transplant, cryotherapy, laser therapy, precision medicine, and combinations thereof.

In certain particular (but non-limiting) embodiments, the patient being treated may have a primary tumor such as (but not limited to) a breast, lung, bladder, skin, intestine, colon, kidney, ovary, pancreas, prostate, brain, stomach, thyroid, head and neck, gastroesophageal tract, connective or other nonepithelial tissue, lymphatic cells, or uterus tumor.

In the methods of the present disclosure, treatment of the cancer may result in one or more of the following: (i) reduction or slowing of tumor metastasis; (ii) prevention or delay of recurrence of the cancer; (iii) extension of disease-free or tumor-free survival time; (iv) increases overall survival time; (v) reduction of the frequency of treatment; (vi) relief of one or more symptoms of the cancer; and/or (vii) reduction of the tumor burden.

When metastasis is present and reduced, the reduced metastasis may be of one or more of the adrenal glands, brain and/or spinal cord, bone, lung, liver and/or pleura, gastrointestinal tract, peritoneum, muscle, lymph nodes, skin, and the like. When metastasis is present or prevented, the primary or secondary tumor of the subject being treated may be a cancer of the breast, lung, bladder, skin, intestine, colon, kidney, ovary, pancreas, prostate, liver, brain, stomach, thyroid, head and neck, gastroesophageal tract, myeloid, lymphoid, connective, or other nonepithelial tissue, uterus, and the like.

In a particular (but non-limiting) embodiment, the primary tumor is breast cancer that is advanced metastatic breast cancer that may be triple negative, and the method further comprises the step of administering histone deacetylase inhibitors (HDACi) to prime the tumor before treatment using the ICI/fuco-ACT combination (such as, but not limited to, a period of priming of about 1 day, 2 days, 3 days, 4 days, 5 days, 6 days, one week, greater than one week, two weeks, greater than two weeks, three weeks, or greater than three weeks). In a particular (but non-limiting) embodiment, the HDACi is administered bi-weekly or tri-weekly for a period of time before treatment using the combination begins.

Certain non-limiting embodiments of the present disclosure are directed to a method of treating cancer or reducing the occurrence or severity of metastasis through improving the trafficking of immune cells to a solid cancer during Adoptive Cell Therapy (ACT), the method comprising the step of administering to the subject having a solid cancer at least one of any of the ICIs disclosed or otherwise contemplated herein and at least one of any of the fuco-ACTs disclosed or otherwise contemplated herein. In certain particular (but non-limiting) embodiments, the ICI(s) and fuco-ACT(s) are present in the same composition; alternatively, the ICI(s) and fuco-ACT(s) are present in separate compositions that are administered simultaneously or wholly or partially sequentially (with either composition being administered first). In addition, each of the compositions may be administered to the patient one or more times.

Certain non-limiting embodiments of the present disclosure relate to a method of enhancing the activity of natural killer (NK), cytotoxic T-cell, or Treg cellular activity in a cancer patient comprising the step of administering at least one of any of the ICIs disclosed or otherwise contemplated herein and at least one of any of the fuco-ACTs disclosed or otherwise contemplated herein. In certain particular (but non-limiting) embodiments, the ICI(s) and fuco-ACT(s) are present in the same composition; alternatively, the ICI(s) and fuco-ACT(s) are present in separate compositions that are administered simultaneously or wholly or partially sequentially (with either composition being administered first). In addition, each of the compositions may be administered to the patient one or more times.

Certain non-limiting embodiments of the present disclosure are related to a method for enhancing antibody-dependent cell-mediated cytotoxicity in a cancer patient comprising the step of administering to the cancer patient at least one of any of the ICIs disclosed or otherwise contemplated herein and at least one of any of the fuco-ACTs disclosed or otherwise contemplated herein. In certain particular (but non-limiting) embodiments, the ICI(s) and fuco-ACT(s) are present in the same composition; alternatively, the ICI(s) and fuco-ACT(s) are present in separate compositions that are administered simultaneously or wholly or partially sequentially (with either composition being administered first). In addition, each of the compositions may be administered to the patient one or more times.

Certain non-limiting embodiments of the present disclosure are directed to a method of treating diseases, disorders, or alleviating or eliminating the symptoms of diseases and disorders, the method comprising the step of administering to a subject in need of treatment at least one of any of the ICIs disclosed or otherwise contemplated herein and at least one of any of the fuco-ACTs disclosed or otherwise contemplated herein, wherein the compounds are administered contemporaneously or within 24 hr of each other. In certain particular (but non-limiting) embodiments, the ICI(s) and fuco-ACT(s) are present in the same composition; alternatively, the ICI(s) and fuco-ACT(s) are present in separate compositions that are administered simultaneously or wholly or partially sequentially (with either composition being administered first). In addition, each of the compositions may be administered to the patient one or more times.

Certain non-limiting embodiments of the present disclosure are directed to a method of improving the cancer cell fighting capacity of immune cells by first contacting the immune cells with a fucosyltransferase and GDP fucose ex vivo such that over 50% of the immune cells so contacted become fucosylated. The immune cells may be any of the immune cells disclosed or otherwise contemplated herein (such as, but not limited to, tumor infiltrating lymphocytes (TILs)). The fucosylation of the TILs may result in: (i) an increased attachment to endothelial cell selectins while in circulation; (ii) the increased surface expression of the trafficking molecule CD162/PSGL-1 and the chemotactic receptor CD183 (CXCR3), and the stimulatory coregulatory molecule CD137 (41BB); (iii) an enhanced cytotoxic T cell (CTL) activity as measured by expression of key components of the cytolytic machinery including at least one of FasL/CD95L, perforin, granzyme, or the ability to kill tumor cells; and/or (iv) an enhanced frequency of fuco-TIL CTLs forming conjugates with target cells compared with nonfucosylated TIL CTLs.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

In the present example, fucosyltransferase-VII (also known as FuT7; TZ 102, Targazyme Inc., Carlsbad, CA) is tested for its ability to fucosylate TILs, restore the expression of sialyl Lewisx (sLex), and enhance TIL binding to E and P selectins.

Ex vivo expanded TILs are suspended in a fucosylation solution comprised of a μg/ml quantity of FuT7 in 1 mmol/L GDP Fucose in PBS with 1% human serum albumin (PBS/HSA). The cell suspension is then incubated at room temperature for 30 minutes. The cells are washed twice in PBS/HSA and then are resuspended in PBS.

The TIL suspension is stained with the FITC-conjugated HECA-452 antibody (BD Biosciences). The HECA-452 antibody targets cutaneous lymphocyte antigen (CLA), shown to be sLeX-positive after fucosylation. TIL fucosylation is confirmed using suitable flow cytometer (for example, a LSR Fortessa; BD Biosciences).

As a result of this procedure, TIL expression of sLeX is increased significantly from baseline levels of 0-5% to 90-100%.

The functional consequences of increased TIL expression of sLeX is measured using a rolling and attachment assay. E-, L-, and P-selectins are attached or immobilized to a suitable substrate (e.g., microscopy chamber). Fucosylated TILs (fuco-TILs) suspended in PBS/HSA are applied to the substrate coated with E-, L-, or P-selectin, allowed to incubate, and then washed with PBS/HSA. Non-fucosylated TILs (nonfuco-TILs) are used as control. TIL attachment is measured using a standard microscopic assay.

### Example 2

In the present example, fuco-TILs from Example 1 are examined for changes in phenotype, specifically for changes in T cell-surface markers, some of which are indicative of activation status and others indicative of enhanced lymphocyte trafficking.

To perform phenotypic analyses, 1.5×10⁶ fuco-TILs and negative control nonfuco-TILs are stained for molecules that modulate T cell trafficking, including CD49d (for example, clone 9F10; BioLegend), CD162 (also known as PSGL-1; for example, clone KPL-1; BioLegend), CD183 (also known as CXCR3; for example, clone 1C6/CXCR3; BD Biosciences), and CD195 (also known as CCR5; for example, clone 2D7/CCR; BD Biosciences), as well as molecules involved in costimulation/inhibition, including CD137 (also known as 41BB; for example, clone 5F4; BioLegend), CD279 (also known as PD1; for example, clone EH12.2H7; BioLegend), and CD357 (also known as GITR; for example, eBioGITR; eBioscience), within 2 hours after fucosylation. The LIVE/DEAD Fixable Aqua Dead Cell Stain Kit (Life Technologies) is used to assess cell viability. Flow cytometry is done on live cells using a suitable flow cytometer (for example, a LSR Fortessa; BD Biosciences). The data are analyzed using an accepted flow cytometry analysis software (for example, FlowJo; FlowJo, LLC).

Differences in cell surface marker expression after fucosylation are determined by cell surface staining and flow cytometry. Fucosylated TILs show increased surface expression of the trafficking molecule CD162/PSGL-1 and the chemotactic receptor CD183 (CXCR3), and the stimulatory coregulatory molecule CD137 (41BB). No other statistically significant changes are detected, including the immune checkpoint receptor CD195 (PD-1) or the regulatory T cell receptor, GITR.

### Example 3

In the present example, fuco-TILs are evaluated for activation status, specifically for cytotoxic T cell (CTL) activation status as measured by expression of key components of the cytolytic machinery (i.e., FasL/CD95L, perforin, and granzyme B), as this is an important feature of successful TIL therapy. TIL (fuco-TIL and nonfuco-TIL) activation is analyzed by measuring the expression of Fas ligand (FasL, also known as CD95L), perforin, and granzyme B. Fuco-TILs and nonfuco-TILs are cocultured with autologous tumor cells obtained from the same host as the TILs, at a ratio of 1:1 overnight at 37° C. At the end of the incubation period, the cells are stained with fluorescently conjugated antibodies targeting CD3, CD8, FasL (BioLegend), CLA, and Ghost Dye Violet 510. After staining for cell surface markers, the cells are permeabilized and stained with fluorescently conjugated antibodies targeting granzyme B and perforin (BioLegend). Staining is analyzed using flow cytometry (BD LSR Fortessa).

The CTL cytolytic machinery after fucosylation is studied by analyzing the intracellular expression of granzyme B and perforin, and surface expression of FasL is studied after coculturing fuco-TILs and nonfuco-TILs with tumor targets. The analysis demonstrates an increase in the percentage of CTLs expressing all three markers after fucosylation. The effect of fucosylation on the binding of CTLs to target cells is also determined by analyzing synapse formation between CTLs and tumor target cells. The data show that fucosylation enhances the frequency of fuco-TIL CTLs forming conjugates with target cells compared with nonfuco-TIL CTLs.

### Example 4

In the present example, fuco-TILs are evaluated for antitumor cytotoxicity against autologous tumor cell targets. To do so, TILs are first passed through a negative selection column (MACS Miltenyi Biotec-CD8 T Cell Isolation Kit) to isolate CD8-postive CTL in the TIL population. These are then processed as described above with TZ 102 to fucosylate the CTL, compared to nonfuco-TILs. Tumor-specific cytotoxicity is then assessed with a standard 4-hour calcein-AM release assay. Tumor target cells are fluorescently labeled with calcein-AM (Invitrogen) for 15 minutes at 37° C. and washed with RPMI-1640 to remove free calcein-AM. These cells are seeded into 60-well Tarasaki plates at multiple effector-to-target (E:T) ratios for 4 hours at 37° C. The reaction is quenched with trypan blue, and fluorescence is measured using a microplate fluorescence reader (BioTek Cytation3). The percent-specific cytotoxicity is calculated using the following formula:

([1-fluorescence target+effector-fluorescence media]:[fluorescence target alone-fluorescence media])×100

Analysis of the data demonstrates that fucosylation increases the antitumor cytotoxic activity of CD8-positive cells within TILs for their corresponding autologous tumor cells. This enhanced tumor killing activity is seen by both increases in absolute tumor cell cytotoxicity as well as a shift in the effector: target ratios necessary for a level of killing (for example, 50% killing) away from higher effector: target ratios needed for nonfuco-TILs to much lower effector: target ratios of fuco-TIL CD*-positive cells needed for the same level of killing (for example, 50% killing).

### Example 5

In the present example, fuco-TILs are compared directly to nonfuco-TILs for their ability to traffic to and infiltrate syngeneic B16-F10 tumors growing in immunocompetent C57BL/6 mice in vivo. The experiment includes a vehicle-treated group which serves as a no-intervention control group for analysis of tumor homing and infiltration. Additionally, a nonfuco-TIL group is included as a comparator for the fuco-TIL group. As a source of B16-F10 TILs, C57BL/6 mice (Charles River Laboratories), eight weeks old, with a body weight (BW) range of 15.4 to 22.0 grams on Day 1 of the study are implanted with B16-F10 tumor cells. Tumors are measured twice per week until the study is ended on Day 10, at which time the tumors are harvested. The TILs are enriched from tumors using standard tumor dissociation and TIL enrichment methods known to the field. After growing for at least 1 week on mice, tumors are harvested and diced into fragments approximately 3-4 mm2. Each tumor fragment is rinsed in fresh media and then plated in individual wells of a 24-well plate in 2 mL of media with 6000IU IL-2/mL. After 3 weeks of expansion, TILs are collected and assayed for T cell phenotypes. Overall, the frequencies of CD4+ and CD8+ T cells are variable.

To evaluate TIL tumor homing and infiltration, C57BL/6 mice are implanted with B16-F10 tumor cells, and the tumors allowed to grow until a mean tumor volume of 100 mm3 is attained. The tumor-bearing mice are randomly sorted into the different treatment groups and one day later are infused with 1×10⁷ fuco-TILs or nonfuco-TILs. Tumors are measured twice per week until the study is ended on Day 10. Each animal is euthanized when its tumor attains the endpoint tumor volume of 1000 mm3 or on the final day of the study, whichever comes first. TILs are enriched from tumors using standard tumor dissociation and TIL enrichment methods known to the field. The TILs are stained with mCD3, mCD4, mCD8 (BioLegend), mCD45, CD90.1 (eBioscience), and Ghost Dye Violet 510 (Tonbo Biosciences), and are analyzed by flow cytometry. CD8-positive T cells obtained from growing B16-F10 tumors are identified as mCD3-positive, mCD8-positive, mCD45-positive, and CD90.1-positive.

Analysis of the data reveals evidence of significantly increased homing of fucosylated B16-F10 tumor-derived TILs into B16-F10 tumors in comparison with nonfuco-TILs.

### Example 6

In the present example, fuco-TILs are compared directly to nonfuco-TILs for their ability to traffic to and infiltrate human triple-negative breast cancer (TNBC) tumors growing in NSG mice in vivo. The experiment includes a vehicle-treated group which serves as a no-intervention control group for analysis of tumor homing and infiltration. Additionally, a nonfuco-TIL group is included as a comparator for the fuco-TIL group. Both the tumor cells and paired autologous TILs are sourced from Yale.

To evaluate TIL tumor homing and infiltration, NSG mice are implanted with human TNBC tumor cells, and the tumors allowed to grow until a mean tumor volume of 100 mm3 is attained. The tumor-bearing mice are randomly sorted into the different treatment groups and one day later are infused with 1×10⁷ human TNBC-derived fuco-TILs or nonfuco-TILs. Tumors are measured twice per week until the study is ended on Day 10. Each animal is euthanized when its tumor attains the endpoint tumor volume of 1000 mm3 or on the final day of the study, whichever comes first. TILs are enriched from tumors using standard tumor dissociation and TIL enrichment methods known to the field. The TILs are stained with hCD3, hCD4, hCD8 (BioLegend), hCD45, hCD90.1 (eBioscience), and Ghost Dye Violet 510 (Tonbo Biosciences), and are analyzed by flow cytometry. CD8-positive T cells obtained from growing B16-F10 tumors are identified as hCD3-positive, hCD8-positive, hCD45-positive, and hCD90.1-positive.

Analysis of the data reveals evidence of significantly increased homing of fucosylated TNBC tumor-derived TILs into TNBC tumors in comparison with nonfuco-TILs.

### Example 7

In the present example, fuco-TILs are compared directly to nonfuco-TILs for antitumor efficacy in the syngeneic, B16-F10 model I immunocompetent mice in vivo. The experiment includes a vehicle-treated group which serves as a no-intervention control group for analysis. Additionally, a nonfuco-TIL group is included as a comparator for the fuco-TIL group. As a source of B16-F10 TILs, C57BL/6 mice (Charles River Laboratories), eight weeks old, with a body weight (BW) range of 15.4 to 22.0 grams on Day 1 of the study are implanted with B16-F10 tumor cells. Tumors are measured twice per week until the study is ended on Day 10, at which time the tumors are harvested. The TILs are enriched from tumors using standard tumor dissociation and TIL enrichment methods known to the field. After growing for at least 1 week on mice, tumors are harvested and diced into fragments approximately 3-4 mm2. Each tumor fragment is rinsed in fresh media and then plated in individual wells of a 24-well plate in 2 mL of media with 6000IU IL-2/mL. After 3 weeks of expansion, TILs are collected and assayed for T cell phenotypes. Overall, the frequencies of CD4+ and CD8+ T cells are variable.

To evaluate B16-F10 TIL antitumor efficacy, C57BL/6 mice are implanted with B16-F10 tumor cells, and the tumors allowed to grow until a mean tumor volume of 100 mm3 is attained. The tumor-bearing mice are randomly sorted into the different treatment groups and one day later (day 1) are infused with 1×10⁷ B16-F10 tumor derived fuco-TILs or nonfuco-TILs. Tumors are measured twice per week until the study is ended. Each animal is euthanized when its tumor attains the endpoint tumor volume of 1500 mm3 and the time to endpoint (TTE) for each mouse was calculated. Treatment response is primarily determined from an analysis of mean tumor volume. Treatment response is secondarily determined from an analysis of percent tumor growth delay (% TGD), defined as the percent increase in the median time to endpoint (TTE) for treated versus control mice, and by log rank significance of differences in survival among groups and regression responses.

Three groups of B16-F10 tumor-bearing C57BL/6 mice are dosed according to the protocol shown in Table 1.

TABLE 1

| Group | Treatment | Route, Frequency |
|---|---|---|
| Group 1 | PBS | i.v., once (day 1) |
| Group 2 | 10 × 10⁷ nonfuco-TILs | i.v., once (day 1) |
| Group 3 | 10 × 10⁷ fuco-TILs | i.v., once (day 1) |

Tumors are measured using calipers twice per week, and each animal is euthanized when its tumor reaches a volume of 1500 mm3 or at the end of the study, whichever comes first. Animals which exit the study for tumor volume endpoint are documented as euthanized for tumor progression (TP), with the date of euthanasia. The time to endpoint (TTE) for analysis is calculated for each mouse by the following equation:

$$\% TGD = [ ( T - C ) \div C ] \times 100$$

where T=median TTE for a treatment group, and C=median TTE for the designated control group.

Treatment efficacy may be determined from the tumor volumes of animals remaining in the study on the last day. The MTV (n) is defined as the median tumor volume on the last day of the study in the number of animals remaining (n) whose tumors had not attained the endpoint volume. Treatment efficacy may also be determined from the incidence and magnitude of regression responses observed during the study. Treatment may cause partial regression (PR) or complete regression (CR) of the tumor in an animal. In a PR response, the tumor volume is 50% or less of its Day 1 volume for three consecutive measurements during the course of the study. In a CR response, the tumor volume is less than the tumor volume on Day 1 of the study. An animal with a CR response at the termination of a study is additionally classified as a tumor-free survivor (TFS). Animals are monitored for regression responses.

Treatment efficacy may also be determined graphically using a “spider plot” to each animal's tumor volume over time. This allows a clear view of the number of animals experiencing the different categories of response, i.e., CR, PR, SD and NR.

For statistical analysis, Prism (GraphPad) is used for graphical presentations and statistical analyses. The log rank test, which evaluates overall survival experience, is used to analyze the significance of the differences between the TTE values of two groups. Log rank analysis includes the data for all animals in a group except those assessed as NTR deaths. Two-tailed statistical analyses are conducted at significance level P=0.05. Group median tumor volumes are plotted as a function of time. When an animal exits the study due to tumor burden, the final tumor volume recorded for the animal is included with the data used to calculate the median volume at subsequent time points. Kaplan-Meier plots show the percentage of animals in each group remaining in the study versus time.

### Graphs:

1. Mean Tumor volume vs. Time, with standard deviation (SD) 2. Individual Tumor Volume vs. Time 3. Kaplan-Meier

### Table:

1. Median TTE and % TGD

In the present example, mice which received fuco-TILs experienced superior tumor regression (as CR), and enhanced tumor control (as PR, SD) vs. mice receiving nonfuco-TILs.

### Example 8

In the present example, fuco-TILs are compared directly to nonfuco-TILs for antitumor efficacy against human TNBC tumors growing in NSG mice in vivo. The experiment includes a vehicle-treated group which serves as a no-intervention control group for analysis. Additionally, a nonfuco-TIL group is included as a comparator for the fuco-TIL group. Both the tumor cells and paired autologous TILs are sourced from Yale. The TILs are enriched from human TNBC tumors using standard tumor dissociation and TIL enrichment methods known to the field.

To evaluate TNBC TIL antitumor efficacy, NSG mice are implanted with human TNBC tumor cells, and the tumors allowed to grow until a mean tumor volume of 100 mm3 is attained. The tumor-bearing mice are randomly sorted into the different treatment groups and one day later are infused with 1×10⁷ human TNBC-derived fuco-TILs or nonfuco-TILs. Tumors are measured twice per week until the study is ended. Each animal is euthanized when its tumor attains the endpoint tumor volume of 1500 mm3 and the time to endpoint (TTE) for each mouse was calculated. Treatment response is primarily determined from an analysis of mean tumor volume. Treatment response is secondarily determined from an analysis of percent tumor growth delay (% TGD), defined as the percent increase in the median time to endpoint (TTE) for treated versus control mice, and by log rank significance of differences in survival among groups and regression responses.

Three groups of TNBC tumor-bearing NSG mice are dosed according to the protocol shown in Table 2.

TABLE 2

| Group | Treatment | Route, Frequency |
|---|---|---|
| Group 1 | PBS | i.v., once (day 1) |
| Group 2 | 10 × 10⁷ nonfuco-TILs | i.v., once (day 1) |
| Group 3 | 10 × 10⁷ fuco-TILs | i.v., once (day 1) |

Tumors are measured using calipers twice per week, and each animal is euthanized when its tumor reaches a volume of 1500 mm3 or at the end of the study, whichever comes first. Animals which exit the study for tumor volume endpoint are documented as euthanized for tumor progression (TP), with the date of euthanasia. The time to endpoint (TTE) for analysis is calculated for each mouse by the following equation:

$$\% TGD = [ ( T - C ) \div C ] \times 100$$

where T=median TTE for a treatment group, and C=median TTE for the designated control group.

Treatment efficacy may be determined from the tumor volumes of animals remaining in the study on the last day. The MTV (n) is defined as the median tumor volume on the last day of the study in the number of animals remaining (n) whose tumors had not attained the endpoint volume. Treatment efficacy may also be determined from the incidence and magnitude of regression responses observed during the study. Treatment may cause partial regression (PR) or complete regression (CR) of the tumor in an animal. In a PR response, the tumor volume is 50% or less of its Day 1 volume for three consecutive measurements during the course of the study. In a CR response, the tumor volume is less than the tumor volume on Day 1 of the study. An animal with a CR response at the termination of a study is additionally classified as a tumor-free survivor (TFS). Animals are monitored for regression responses.

Treatment efficacy may also be determined graphically using a “spider plot” to each animal's tumor volume over time. This allows a clear view of the number of animals experiencing the different categories of response, i.e., CR, PR, SD and NR.

For statistical analysis, Prism (GraphPad) is used for graphical presentations and statistical analyses. The log rank test, which evaluates overall survival experience, is used to analyze the significance of the differences between the TTE values of two groups. Log rank analysis includes the data for all animals in a group except those assessed as NTR deaths. Two-tailed statistical analyses are conducted at significance level P=0.05. Group median tumor volumes are plotted as a function of time. When an animal exits the study due to tumor burden, the final tumor volume recorded for the animal is included with the data used to calculate the median volume at subsequent time points. Kaplan-Meier plots show the percentage of animals in each group remaining in the study versus time.

### Graphs:

1. Mean Tumor volume vs. Time, with standard deviation (SD) 2. Individual Tumor Volume vs. Time 3. Kaplan-Meier

### Table:

1. Median TTE and % TGD

In the present example, mice which received fuco-TILs experienced tumor regression (as CR), and enhanced tumor control (as PR, SD) vs. mice receiving nonfuco-TILs.

### Example 9

In the present example, fuco-TILs are compared directly to nonfuco-TILs for antitumor efficacy in the syngeneic, B16-F10 model in immunocompetent mice in vivo. Furthermore, the effect of adding an immune checkpoint inhibitor (ICI) antibody in combination with fuco-TILs is included to evaluate the potential for additive or even synergistic antitumor activity for this combination. The experiment includes both a vehicle-treated group and an ICI-only group which serve as control group for analysis. Additionally, a nonfuco-TIL group is included as a comparator for the fuco-TIL group, and a nonfuco-TIL plus ICI group is included as a comparator for the fuco-TIL plus ICI group. As a source of B16-F10 TILs, C57BL/6 mice (Charles River Laboratories), eight weeks old, with a body weight (BW) range of 15.4 to 22.0 grams on Day 1 of the study are implanted with B16-F10 tumor cells. Tumors are measured twice per week until the study is ended on Day 10, at which time the tumors are harvested. The TILs are enriched from tumors using standard tumor dissociation and TIL enrichment methods known to the field. After growing for at least 1 week on mice, tumors are harvested and diced into fragments approximately 3-4 mm2. Each tumor fragment is rinsed in fresh media and then plated in individual wells of a 24-well plate in 2 mL of media with 6000IU IL-2/mL. After 3 weeks of expansion, TILs are collected and assayed for T cell phenotypes. Overall, the frequencies of CD4+ and CD8+ T cells are variable.

To evaluate B16-F10 TIL antitumor efficacy, C57BL/6 mice are implanted with B16-F10 tumor cells, and the tumors allowed to grow until a mean tumor volume of 100 mm3 is attained. The tumor-bearing mice are randomly sorted into the different treatment groups and one day later (day 1) are infused with 1×10⁷ B16-F10 tumor derived fuco-TILs or nonfuco-TILs. Tumors are measured twice per week until the study is ended. Each animal is euthanized when its tumor attains the endpoint tumor volume of 1500 mm3 and the time to endpoint (TTE) for each mouse was calculated. Treatment response is primarily determined from an analysis of mean tumor volume. Treatment response is secondarily determined from an analysis of percent tumor growth delay (% TGD), defined as the percent increase in the median time to endpoint (TTE) for treated versus control mice, and by log rank significance of differences in survival among groups and regression responses.

Six groups of B16-F10 tumor-bearing C57BL/6 mice are dosed according to the protocol shown Table 3. As described above, both fuco-TILs and nonfuco-TILs are dosed i.v. at 10×10⁷ per mouse. The ICI antibody in this study is an anti-mouse PD-1, dosed at 5 mg/kg, i.p. biweekly×3.

TABLE 3

| Group | Treatment | Route, Frequency |
|---|---|---|
| Group 1 | PBS | i.v., once (day 1) |
| Group 2 | nonfuco-TILs | i.v., once (day 1) |
| Group 3 | fuco-TILs | i.v., once (day 1) |
| Group 4 | PD-1 antibody | i.p., biwk × 3 |
| Group 5 | nonfuco-TILs + PD-1 antibody | i.v., once (day 1) |
| Group 6 | fuco-TILs + PD-1 antibody | i.v., once (day 1) |

Tumors are measured using calipers twice per week, and each animal is euthanized when its tumor reaches a volume of 1500 mm3 or at the end of the study, whichever comes first. Animals which exit the study for tumor volume endpoint are documented as euthanized for tumor progression (TP), with the date of euthanasia. The time to endpoint (TTE) for analysis is calculated for each mouse by the following equation:

$$\% TGD = [ ( T - C ) \div C ] \times 100$$

where T=median TTE for a treatment group, and C=median TTE for the designated control group.

Treatment efficacy may be determined from the tumor volumes of animals remaining in the study on the last day. The MTV (n) is defined as the median tumor volume on the last day of the study in the number of animals remaining (n) whose tumors had not attained the endpoint volume. Treatment efficacy may also be determined from the incidence and magnitude of regression responses observed during the study. Treatment may cause partial regression (PR) or complete regression (CR) of the tumor in an animal. In a PR response, the tumor volume is 50% or less of its Day 1 volume for three consecutive measurements during the course of the study. In a CR response, the tumor volume is less than the tumor volume on Day 1 of the study. An animal with a CR response at the termination of a study is additionally classified as a tumor-free survivor (TFS). Animals are monitored for regression responses.

Treatment efficacy may also be determined graphically using a “spider plot” to each animal's tumor volume over time. This allows a clear view of the number of animals experiencing the different categories of response, i.e., CR, PR, SD and NR.

For statistical analysis, Prism (GraphPad) is used for graphical presentations and statistical analyses. The log rank test, which evaluates overall survival experience, is used to analyze the significance of the differences between the TTE values of two groups. Log rank analysis includes the data for all animals in a group except those assessed as NTR deaths. Two-tailed statistical analyses are conducted at significance level P=0.05. Group median tumor volumes are plotted as a function of time. When an animal exits the study due to tumor burden, the final tumor volume recorded for the animal is included with the data used to calculate the median volume at subsequent time points. Kaplan-Meier plots show the percentage of animals in each group remaining in the study versus time.

### Graphs:

1. Mean Tumor volume vs. Time, with standard deviation (SD)
2. Individual Tumor Volume vs. Time
3. Kaplan-Meier

### Table:

### 1. Median TTE and % TGD

In the present example, mice which received fuco-TILs again experienced superior tumor regression (as CR), and enhanced tumor control (as PR, SD) vs. mice receiving nonfuco-TILs. The addition of ICI modestly enhanced the activity of nonfuco-TILs, however, ICI addition to fuco-TILs led to complete responses (tumor regression) in all mice so treated.

### Example 10

In the present example, fuco-TILs are compared directly to nonfuco-TILs for antitumor efficacy against human TNBC tumors growing in NSG mice in vivo. The experiment includes a vehicle-treated group which serves as a no-intervention control group for analysis. Additionally, a nonfuco-TIL group is included as a comparator for the fuco-TIL group. Furthermore, the effect of adding an immune checkpoint inhibitor (ICI) antibody in combination with fuco-TILs is included to evaluate the potential for additive or even synergistic antitumor activity for this combination. The experiment includes both a vehicle-treated group and an ICI-only group which serve as control group for analysis. Additionally, a nonfuco-TIL group is included as a comparator for the fuco-TIL group, and a nonfuco-TIL plus ICI group is included as a comparator for the fuco-TIL plus ICI group. Both the tumor cells and paired autologous TILs are sourced from Yale. The TILs are enriched from human TNBC tumors using standard tumor dissociation and TIL enrichment methods known to the field.

To evaluate TNBC TIL antitumor efficacy, NSG mice are implanted with human TNBC tumor cells, and the tumors allowed to grow until a mean tumor volume of 100 mm3 is attained. The tumor-bearing mice are randomly sorted into the different treatment groups and one day later are infused with 1×10⁷ human TNBC-derived fuco-TILs or nonfuco-TILs. Tumors are measured twice per week until the study is ended. Each animal is euthanized when its tumor attains the endpoint tumor volume of 1500 mm3 and the time to endpoint (TTE) for each mouse was calculated. Treatment response is primarily determined from an analysis of mean tumor volume. Treatment response is secondarily determined from an analysis of percent tumor growth delay (% TGD), defined as the percent increase in the median time to endpoint (TTE) for treated versus control mice, and by log rank significance of differences in survival among groups and regression responses.

Six groups of TNBC tumor-bearing NSG mice are dosed according to the protocol shown in Table 4.

As described above, both fuco-TILs and nonfuco-TILs are dosed i.v. at 10×10⁷ per mouse. The ICI antibody in this study is an anti-mouse PD-1, dosed at 5 mg/kg, i.p. biweekly×3.

TABLE 4

| Group | Treatment | Route, Frequency |
|---|---|---|
| Group 1 | PBS | i.v., once (day 1) |
| Group 2 | nonfuco-TILs | i.v., once (day 1) |
| Group 3 | fuco-TILs | i.v., once (day 1) |
| Group 4 | PD-1 antibody | i.p., biwk × 3 |
| Group 5 | nonfuco-TILs + PD-1 antibody | i.v., once (day 1) |
| Group 6 | fuco-TILs + PD-1 antibody | i.v., once (day 1) |

Tumors are measured using calipers twice per week, and each animal is euthanized when its tumor reaches a volume of 1500 mm3 or at the end of the study, whichever comes first. Animals which exit the study for tumor volume endpoint are documented as euthanized for tumor progression (TP), with the date of euthanasia. The time to endpoint (TTE) for analysis is calculated for each mouse by the following equation:

$$\% TGD = [ ( T - C ) \div C ] \times 100$$

where T=median TTE for a treatment group, and C=median TTE for the designated control group.

Treatment efficacy may be determined from the tumor volumes of animals remaining in the study on the last day. The MTV (n) is defined as the median tumor volume on the last day of the study in the number of animals remaining (n) whose tumors had not attained the endpoint volume. Treatment efficacy may also be determined from the incidence and magnitude of regression responses observed during the study. Treatment may cause partial regression (PR) or complete regression (CR) of the tumor in an animal. In a PR response, the tumor volume is 50% or less of its Day 1 volume for three consecutive measurements during the course of the study. In a CR response, the tumor volume is less than the tumor volume on Day 1 of the study. An animal with a CR response at the termination of a study is additionally classified as a tumor-free survivor (TFS). Animals are monitored for regression responses.

Treatment efficacy may also be determined graphically using a “spider plot” to each animal's tumor volume over time. This allows a clear view of the number of animals experiencing the different categories of response, i.e., CR, PR, SD and NR.

For statistical analysis, Prism (GraphPad) is used for graphical presentations and statistical analyses. The log rank test, which evaluates overall survival experience, is used to analyze the significance of the differences between the TTE values of two groups. Log rank analysis includes the data for all animals in a group except those assessed as NTR deaths. Two-tailed statistical analyses are conducted at significance level P=0.05. Group median tumor volumes are plotted as a function of time. When an animal exits the study due to tumor burden, the final tumor volume recorded for the animal is included with the data used to calculate the median volume at subsequent time points. Kaplan-Meier plots show the percentage of animals in each group remaining in the study versus time.

### Graphs:

1. Mean Tumor volume vs. Time, with standard deviation (SD) 2. Individual Tumor Volume vs. Time 3. Kaplan-Meier

### Table:

1. Median TTE and % TGD

In the present example, mice which received fuco-TILs again experienced superior tumor regression (as CR), and enhanced tumor control (as PR, SD) vs. mice receiving nonfuco-TILs. The addition of ICI modestly enhanced the activity of nonfuco-TILs, however, ICI addition to fuco-TILs led to complete responses (tumor regression) in all mice so treated.

### Example 11

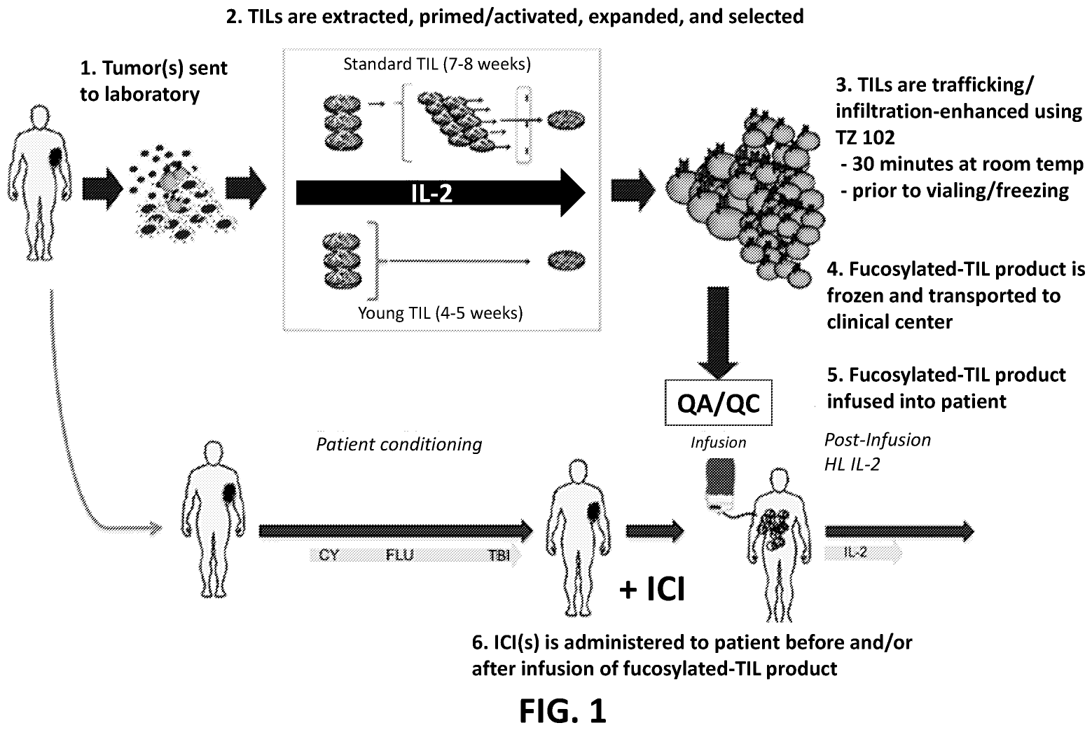

FIG. 1 graphically depicts one non-limiting embodiment of a combinatorial therapy method constructed in accordance with the present disclosure, wherein the combinatorial therapy includes the use of immune checkpoint inhibitors with tumor infiltrating lymphocytes that have been fucosylated ex vivo.

In the method, at least a portion of at least one tumor is excised from a patient and sent to a laboratory for processing. Immune cells such as (but not limited to) tumor-infiltrating leukocytes (TILs) are extracted from the tumor and may be exposed to one or more optional steps, such as (but not limited to) priming/activation, expansion, and/or selection steps (such as, but not limited to, exposure to IL-2). Then these cells are exposed to TZ 102 (FUT7) for at least about 30 minutes at room temperature, which fucosylates the cells to provide fuco-ACT and thereby enhances trafficking and tumor infiltration characteristics of the cells. The fuco-ACT may be directly transported to a clinical center for infusion into a patient; optionally, the fuco-ACT may be frozen for transportation and/or storage purposes prior to delivery to the clinical center. Upon arrival at the clinical center, the fuco-ACT are infused back into the patient. Then one or more ICIs may be administered to the patient, either prior to, concurrently with, or following administration of the fuco-ACT.

Prior to the infusion step, the patient may optionally be conditioned for transplantation of the fuco-ACT by one or more chemotherapeutic methods known in the art, such as (but not limited to) administration of cyclophosphamide (CY), fludarabine (FLU), and/or total body irradiation (TBI). In addition, following infusion of the fuco-ACT, the patient may optionally receive an immunotherapeutic dosage of high dose IL-2 (HD IL-2).

### Example 12

Several cancer immunotherapy breakthroughs have been found in the last several years, including the stimulation of immune cell growth signals (such as, but not limited to, with CD28, IL-2, and/or interferon (IFN); the removal of immune suppression with checkpoint inhibitors (such as, but not limited to, inhibitors of CTLA-4 and the PD1: PD-L1 interaction); and adoptive cell therapy (such as, but not limited to, with CAR-T cells, TCR-T cells, TILs, NK cells, dendritic cells, etc).

However, while immunotherapy is promising, challenges remain. Immune cells do not traffic to and infiltrate into solid tumors well; indeed, less than 3% of immune cells reach tumors and infiltrate to the tumor core. Immune checkpoint inhibitors have limited efficacy for most cancer patients with solid tumors. Immune checkpoint inhibitor (ICI)-mediated antitumor responses depend on the infiltration of T cells capable of recognizing and killing tumor cells; however, ICIs are not effective in “cold” tumors, which have been characterized as having a lack or poor T-cell infiltration. “Cold” tumors include immune-excluded tumors (i.e., CD8+ T cells localize only at invasion margins and do not efficiently infiltrate the tumor) and immune-desert tumors (i.e., CD8+ T cells are absent from the tumor and its periphery). In contrast, “hot” tumors refer to immune-inflamed tumors characterized by high immune cell infiltration and increased IFNγ signaling, as well as high (relative) PD-L1 expression. In contrast, adoptive immune cell therapy does not work for most cancer patients with solid tumors; only 3 to 5% of introduced T-cells reach the tumor microenvironment.

Cancer immunotherapies that include TIL therapies and/or checkpoint inhibitors are promising; however, the efficacy in cold cancer tumor indications is limited, as shown in Table 5 below.

TABLE 5

| Indication | ICI (ORR) | TILs (ORR) | ICI + TILs (ORR) |
|---|---|---|---|
| Pancreatic Cancer | 3.1% | NYA | NYA |
| Prostate Cancer | 5% | NYA | NYA |
| Ovarian Cancer | 8.0% | 15.0% | NYA |
| Head & Neck Cancer | 13.3% | 21.4% | 38.9% |
| Breast Cancer | 18.5% | 24.0% | NYA |
| Liver Cancer | 20% | 36.0% | NYA |
| Melanoma | 46% | 38% | NYA |
| NSCLC | 44.8% | 21.4% | NYA |

*ORR = tumor objective response rate;
NYA = Not Yet Available (Ongoing trials)

Immune checkpoint inhibitors are most responsive to hot tumors than cold tumors.

Therefore, one of the goals of the present disclosure is to turn cold tumors into hot tumors.

The present disclosure has found that fucosylation of TILs ex vivo results in increased immune cell priming/activation, increased immune cell expansion, increased immune cell trafficking to tumors, and increased infiltration of tumors; these results can turn cold tumors into hot tumors, thereby immune-potentiating checkpoint inhibitors. In particular, ex vivo fucosylated TILs (for example, TILs isolated from the patient, a donor, or iPSCs, or genetically engineered cells and then exposed to TZ 102 (or other fucosyltransferase) and a fucose donor) are activated during the manufacturing process and expanded with cell selection during the manufacturing process. These ex vivo fucosylated TILs shown a 2.5-fold improved trafficking to and infiltration into tumors. In addition, ex vivo fucosylated TIL manufacturing results in the delivery of several antitumor effector cell types with antigen receptor diversity for improved killing of solid tumors. Further, the effects observed are seen with more than just CD8+ effector cells; ex vivo fucosylation also facilitates the trafficking and infiltration of various cancer kill cells (such as, but not limited to, NK cells, dendritic cells, B cells, etc.) into the tumors, thereby improving total efficacy.

Approximately 200% to 500% more ex vivo fucosylated immune cells will enter the tumor micro-environment and infiltrate tumors, thereby increasing the cancer fighting ability of these trafficked/infiltrated immune cells and also improving the efficacy of concomitantly administered immune checkpoint inhibitors.

The specific cell surface modification of fucosylation has been shown to increase T-cell tumor targeting and effectiveness. Fucosylation is a post-translation modification that “turns on” T-cell homing and infiltration. Fucosylation enables T-cells to stick to and cross blood vessel walls, thereby penetrating blood vessel walls as well as the tumor mass.

For example (but not by way of limitation), exposure of TCR-T cells to TZ 102 increased the fucosylation level from 25% to 92%. Then when measuring TCR-T homing and penetration in an A549 lung cancer model, 200% more TZ 102-treated T-cells trafficked to the lung tumors, demonstrating that fucosylation doubled the number of TCR-T cells that infiltrated the tumor. Further, fucosylation increased the percentage of both CD3+ and CD8+ TCR-T cells in the tumor by 3.3× and 4.7×, respectively. This demonstrates that TZ102-treated TCR-T cells traffic to and infiltrate into lung cancers 330-470% more effectively than T-cells that were not treated with TZ102.

Al-Atrash et al. (Clinical Cancer Research (2019) 25 (8): 2610-2620) demonstrated that 200% more TZ 102-treated T-cells trafficked to and infiltrated breast tumors in an SKBR3 breast cancer model, with 70% reduction in breast tumors compared to T-cells that were not treated with TZ102. This reference also demonstrated that 250% more TZ102-treated T-cells trafficked to and infiltrated melanoma in a B16-F10 melanoma animal model, with a 77% reduction in melanoma cells compared to T-cells that were not treated with TZ102. Fucosylation increased TME homing, penetration, and anti-tumor cytotoxicity, leading to increased TCR-T cell antitumor efficacy.

### Example 13

A Phase 2a, 2:1 randomized, double-blind study to demonstrate the safety and efficacy of TZ102-fucosylated TILs in combination with immune checkpoint inhibitors (ICIs) for Stage III & IV metastatic melanoma patients is conducted. About 78,000 cases of Stage III & IV metastatic melanoma cases are diagnosed per year, with over 9,000 deaths per year. The 5-year survival rate is 5-19%, with a median overall survival of 5.3 months. Therefore, there is an unmet medical need for treating these patients.

Up to 33 subjects with Stage III or IV unresectable melanoma patients whose cancer has progressed despite immune checkpoint therapy and/or BRAF/MEK inhibitors (if they have BRAF mutations) are included. Up to 20 subjects receive the combination TZ102-TILs+ICI therapy, up to 3 subjects receive TZ102-TIL therapy alone, and up to 10 subjects receive Standard of Care+ICI therapy. A lymphocyte depleting preconditioning regimen is included before the infusion of autologous TZ102-fucosylated TILs, and a regimen of IL-2 is administered post-infusion.

The efficacy endpoints are measured primarily as objective response rate (ORR) and secondarily as duration of response, progression free survival, and overall survival. Safety endpoints are also evaluated based on the number and rate of all adverse and serious events (AEs) and related AEs categorized by severity, as well as the number and rate of clinically significant abnormal laboratory values.

This study demonstrates the synergistic combination of TZ102-fucosylated TILs and ICIs, as well as the safety and efficacy of TZ102-fucosylated TILs in combination with ICIs for Stage III & IV metastatic melanoma patients.

### Example 14

A Phase 2a, 2:1 randomized, double-blind study to demonstrate the safety and efficacy of TZ102-fucosylated TILs in combination with immune checkpoint inhibitors (ICIs) for patients with metastatic breast cancer is conducted. Metastatic breast cancer is the second-leading cause of cancer death worldwide, with about 284,200 new cases per year, and over 44,000 deaths per year. The 5-year survival rate is about 11%, with a median overall survival of 12-18 months. Therefore, there is an unmet medical need for treating these patients.

Up to 33 subjects with pretreated metastatic triple negative breast cancer whose cancer has progressed despite the standard of care are included. Up to 20 subjects receive the combination TZ102-TILs+ICI therapy, up to 3 subjects receive the single arm TZ102-TIL therapy alone, and up to 10 subjects receive Standard of Care+ICI therapy. A lymphocyte depleting preconditioning regimen is included before the infusion of autologous TZ102-fucosylated TILs, and a regimen of IL-2 is administered post-infusion.

The efficacy endpoints are measured primarily as objective response rate (ORR) and secondarily as duration of response, progression free survival, and overall survival. Safety endpoints are also evaluated to characterize the safety profile of the TIL as a single therapy in metastatic triple negative breast cancer patients as measured by incidence of Grade ≥3 treatment-emergent adverse events (TEAEs).

This study demonstrates the synergistic combination of TZ102-fucosylated TILs and ICIs, as well as the safety and efficacy of TZ102-fucosylated TILs in combination with ICIs for metastatic breast cancer.

### Non-Limiting Illustrative Embodiments

Illustrative embodiment 1. An adoptive cell therapeutic pharmaceutical composition, comprising: at least one isolated immune cell type that has been fucosylated ex vivo; and at least one immune checkpoint inhibitor.

Illustrative embodiment 1a. The composition of illustrative embodiment 1, further comprising at least one pharmaceutically acceptable carrier.

Illustrative embodiment 2. The composition of illustrative embodiment 1 or 1a, wherein the at least one isolated immune cell type is selected from the group consisting of cytotoxic T cells (CTL), regulatory T cells (Treg), helper T cells, NK cells, B cells, dendritic cells, genetically modified versions thereof, and combinations thereof.

Illustrative embodiment 2a. The composition of illustrative embodiment 2, wherein the genetically modified version of the immune cell type is selected from the group consisting of chimeric antigen receptor gene-transduced T-cells (CAR-T cells), T-cell receptor gene-transduced T cells (TCR-T cells), chimeric antigen receptor gene-transduced natural killer (NK) cells (CAR-NK cells), and combinations thereof.

Illustrative embodiment 3. The composition of illustrative embodiment 1 or 2, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPa inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

Illustrative embodiment 4. The composition of illustrative embodiment 3, wherein the at least one immune checkpoint inhibitor comprises at least one PD-1 inhibitor and at least one LAG-3 inhibitor.

Illustrative embodiment 4a. The composition of any one of illustrative embodiments 1-4, wherein at least one of: the composition comprises a therapeutically effective amount of the at least one immune checkpoint inhibitor; and/or the composition comprises a therapeutically effective amount of the at least one isolated immune cell that has been fucosylated ex vivo.

Illustrative embodiment 4b. The composition of any one of illustrative embodiments 1-4a, wherein the at least one isolated immune cell type that has been fucosylated ex vivo is produced by a method comprising the steps of: 1) identifying a cancer patient in need of adoptive cell therapy; 2) isolating immune cells from the patient's blood or cancer tumor; 3) expanding the immune cells ex vivo; and 4) contacting the expanded immune cells with a fucosyltransferase and at least one of fucose or GDP-fucose to provide fucosylated immune cells.

Illustrative embodiment 4c. The composition of illustrative embodiment 4b, wherein the fucose transferase (FUT) is selected from the group consisting of FUT1, FUT2, FUT3, FUT4, FUT5, FUT6, FUT7, FUT8, FUT9, FUT10, FUT11, and combinations thereof.

Illustrative embodiment 4d. The composition of illustrative embodiment 4c, wherein the fucose transferase is selected from the group consisting of FUT3, FUT5, FUT6, FUT7, FUT9, FUT10, and combinations thereof.

Illustrative embodiment 4e. The composition of any one of illustrative embodiments 4b-4d, wherein the method further comprises, after step (4), the step of (4a) separating fucosylated lymphocytes from other components after step (4), and wherein step (5) is further defined as returning the fucosylated lymphocytes to the patient intravenously.

Illustrative embodiment 4f. The composition of illustrative embodiment 4e, wherein the method further comprises, after step (4a), the step of (4b) isolating specific fucosylated lymphocyte types from other fucosylated lymphocytes, and wherein step (5) is further defined as returning the specific fucosylated lymphocyte types to the patient intravenously.

Illustrative embodiment 5. A system, comprising: a composition comprising at least one immune checkpoint inhibitor (ICI); and a composition comprising at least one fucosylated immune cell type for adoptive cell therapy (fuco-ACT).

Illustrative embodiment 6. The system of illustrative embodiment 5, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPa inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

Illustrative embodiment 7. The system of illustrative embodiment 6, wherein the at least one immune checkpoint inhibitor comprises at least one PD-1 inhibitor and at least one LAG-3 inhibitor.

Illustrative embodiment 8. The system of illustrative embodiment 6 or 7, wherein the at least one immune cell type is selected from the group consisting of cytotoxic T cells (CTL), regulatory T cells (Treg), helper T cells, NK cells, B cells, dendritic cells, genetically modified versions thereof, and combinations thereof.

Illustrative embodiment 8a. The system of illustrative embodiment 8, wherein the genetically modified version of the immune cell type is selected from the group consisting of chimeric antigen receptor gene-transduced T-cells (CAR-T cells), T-cell receptor gene-transduced T cells (TCR-T cells), chimeric antigen receptor gene-transduced natural killer (NK) cells (CAR-NK cells), and combinations thereof.

Illustrative embodiment 8b. The system of any one of illustrative embodiments 6-8a, wherein the composition provides a therapeutically effective amount of immune checkpoint inhibitor.

Illustrative embodiment 8c. The system of any one of illustrative embodiments 6-8b, wherein the composition provides a therapeutically effective amount of fuco-ACT.

Illustrative embodiment 8d. The system of any one of illustrative embodiments 6-8c, wherein the system is further defined as a kit.

Illustrative embodiment 8e. The system of any one of illustrative embodiments 6-8d, wherein the fuco-ACT is produced by a method comprising the steps of: 1) identifying a cancer patient in need of adoptive cell therapy; 2) isolating immune cells from the patient's blood or cancer tumor; 3) expanding the immune cells ex vivo; and 4) contacting the expanded immune cells with a fucosyltransferase and at least one of fucose or GDP-fucose to provide fucosylated immune cells.

Illustrative embodiment 8f. The system of illustrative embodiment 8e, wherein the fucose transferase (FUT) is selected from the group consisting of FUT1, FUT2, FUT3, FUT4, FUT5, FUT6, FUT7, FUT8, FUT9, FUT10, FUT11, and combinations thereof.

Illustrative embodiment 8g. The system of illustrative embodiment 8f, wherein the fucose transferase is selected from the group consisting of FUT3, FUT5, FUT6, FUT7, FUT9, FUT10, and combinations thereof.

Illustrative embodiment 9. An adoptive cell therapy method, comprising the steps of: 1) identifying a cancer patient in need of adoptive cell therapy; 2) isolating immune cells from the patient's blood or cancer tumor; 3) expanding the immune cells ex vivo; 4) contacting the expanded immune cells with a fucosyltransferase and at least one of fucose or GDP-fucose to provide fucosylated immune cells; 5) returning the fucosylated immune cells to the patient intravenously; and 6) administering at least one immune checkpoint inhibitor to the patient simultaneously or wholly or partially sequentially with the fucosylated immune cells.

Illustrative embodiment 10. The method of illustrative embodiment 9, further comprising, after step (4), the step of (4a) separating fucosylated lymphocytes from other components after step (4), and wherein step (5) is further defined as returning the fucosylated lymphocytes to the patient intravenously.

Illustrative embodiment 11. The method of illustrative embodiment 10, further comprising, after step (4a), the step of (4b) isolating specific fucosylated lymphocyte types from other fucosylated lymphocytes, and wherein step (5) is further defined as returning the specific fucosylated lymphocyte types to the patient intravenously.

Illustrative embodiment 12. The method of any one of illustrative embodiments 9-11, wherein the fucose transferase (FUT) is selected from the group consisting of FUT1, FUT2, FUT3, FUT4, FUT5, FUT6, FUT7, FUT8, FUT9, FUT10, FUT11, and combinations thereof.

Illustrative embodiment 12a. The method of illustrative embodiment 12, wherein the fucose transferase is selected from the group consisting of FUT3, FUT5, FUT6, FUT7, FUT9, FUT10, and combinations thereof.

Illustrative embodiment 12b. The method of any one of illustrative embodiments 9-12a, wherein at least step (6) is repeated one or more times.

Illustrative embodiment 12c. The method of any one of illustrative embodiments 9-12b, wherein at least step (5) is repeated one or more times.

Illustrative embodiment 12d. The method of any one of illustrative embodiments 9-12c, wherein the at least one immune checkpoint inhibitor is administered prior to the at least one fucosylated immune cell type for adoptive cell therapy (fuco-ACT).

Illustrative embodiment 12e. The method of any one of illustrative embodiments 9-12d, wherein the at least one immune checkpoint inhibitor is administered concurrently with the at least one fucosylated immune cell type for adoptive cell therapy (fuco-ACT).

Illustrative embodiment 12f. The method of any one of illustrative embodiments 9-12e, wherein the at least one fucosylated immune cell type for adoptive cell therapy (fuco-ACT) is administered prior to the at least one immune checkpoint inhibitor.

Illustrative embodiment 13. A method of treating one or more cancers and reducing the occurrence or severity of metastasis in a cancer patient, the method comprising the step of: administering to the cancer patient, either simultaneously or wholly or partially sequentially, a combination of at least one ICI and at least one fucosylated immune cell type for adoptive cell therapy (fuco-ACT).

Illustrative embodiment 13a. The method of illustrative embodiment 13, wherein the at least one ICI and at least one fucosylated immune cell type for adoptive cell therapy (fuco-ACT) are present in the same pharmaceutical composition.

Illustrative embodiment 13b. The method of illustrative embodiment 13, wherein the at least one ICI and at least one fucosylated immune cell type for adoptive cell therapy (fuco-ACT) are present in separate pharmaceutical compositions.

Illustrative embodiment 14. The method of any one of illustrative embodiments 9-13b, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPa inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

Illustrative embodiment 15. The method of any one of illustrative embodiments 9-14, wherein the at least one immune checkpoint inhibitor comprises at least one PD-1 inhibitor and at least one LAG-3 inhibitor.

Illustrative embodiment 16. The method of any one of illustrative embodiments 9-15, wherein the at least one immune cell type is selected from the group consisting of cytotoxic T cells (CTL), regulatory T cells (Treg), helper T cells, NK cells, B cells, dendritic cells, genetically modified versions thereof, and combinations thereof.

Illustrative embodiment 16a. The method of illustrative embodiment 16, wherein the genetically modified version of the immune cell type is selected from the group consisting of chimeric antigen receptor gene-transduced T-cells (CAR-T cells), T-cell receptor gene-transduced T cells (TCR-T cells), chimeric antigen receptor gene-transduced natural killer (NK) cells (CAR-NK cells), and combinations thereof.

Illustrative embodiment 17. The method of any one of illustrative embodiments 9-16, wherein the cancer is selected from the group consisting of prostate cancer, skin cancer, ovarian cancer, breast cancer, a non-lymphoid parenchymal organ cancer; a cancer of the head and/or neck; leukemia; a cancer of the retina; a cancer of the esophagus; multiple myeloma; melanoma; colorectal cancer; lung cancer; cervical cancer; endometrium carcinoma; gallbladder cancer; liver cancer; thyroid follicular cancer; gastric cancer; non-small cell lung carcinoma; glioma; urothelial cancer; bladder cancer; prostate cancer; renal cell cancer; infiltrating ductal carcinoma; glioblastoma multiform; and combinations thereof.

Illustrative embodiment 18. The method of any one of illustrative embodiments 9-17, further comprising the step of administering at least one additional treatment to the cancer patient, wherein the at least one additional treatment is selected from the group consisting of radiation, surgery, chemotherapy, immunotherapy, targeted therapy, hormone therapy, stem cell transplant, cryotherapy, laser therapy, precision medicine, and combinations thereof.

Illustrative embodiment 18a. The method of any one of illustrative embodiments 9-18, wherein administration of the at least one ICI is repeated one or more times.

Illustrative embodiment 18b. The method of any one of illustrative embodiments 9-18a, wherein administration of the at least one fucosylated immune cell type for adoptive cell therapy (fuco-ACT) is repeated one or more times.

Illustrative embodiment 18c. The method of any one of illustrative embodiments 9-18b, wherein the at least one immune checkpoint inhibitor is administered prior to the at least one fucosylated immune cell type for adoptive cell therapy (fuco-ACT).

Illustrative embodiment 18d. The method of any one of illustrative embodiments 9-18c, wherein the at least one immune checkpoint inhibitor is administered concurrently with the at least one fucosylated immune cell type for adoptive cell therapy (fuco-ACT).

Illustrative embodiment 18e. The method of any one of illustrative embodiments 9-18d, wherein the at least one fucosylated immune cell type for adoptive cell therapy (fuco-ACT) is administered prior to the at least one immune checkpoint inhibitor.

Illustrative embodiment 19. The method of any one of illustrative embodiments 9-18e, wherein the patient has a primary tumor selected from the group consisting of breast, lung, bladder, skin, intestine, colon, kidney, ovary, pancreas, prostate, brain, stomach, thyroid, head and neck, gastroesophageal tract, connective or other nonepithelial tissue, lymphatic cells, or uterus tumor.

Illustrative embodiment 20. The method of illustrative embodiment 19, wherein the primary tumor is breast cancer that is advanced metastatic breast cancer and optionally triple negative, and wherein the method further comprises the step of administering histone deacetylase inhibitors to the patient to prime the tumor from about 1 day to about 3 weeks before treatment with the ICI/fuco-ACT combination.

Illustrative embodiment 21. The method of any one of illustrative embodiments 9-20, wherein treatment of the cancer results in one or more of the following: (i) reduction or slowing of tumor metastasis; (ii) prevention or delay of recurrence of the cancer; (iii) extension of disease-free or tumor-free survival time; (iv) increases overall survival time; (v) reduction of the frequency of treatment; (vi) relief of one or more symptoms of the cancer and; (vii) reduction of the tumor burden.

Illustrative embodiment 22. The method of any one of illustrative embodiments 13-21, wherein the metastasis that is reduced is metastasis of one or more of the adrenal glands, brain and/or spinal cord, bone, lung, liver and/or pleura, gastrointestinal tract, peritoneum, muscle, lymph nodes and skin.

Illustrative embodiment 23. The method of any one of illustrative embodiments 13-22, wherein the reduction or prevention of metastasis is where the primary or secondary tumor of the subject being treated is a cancer of the breast, lung, bladder, skin, intestine, colon, kidney, ovary, pancreas, prostate, liver, brain, stomach, thyroid, head and neck, gastroesophageal tract, myeloid, lymphoid, connective, or other nonepithelial tissue, and uterus.

Illustrative embodiment 24. The method of any one of illustrative embodiments 9-23, wherein the cancer is triple negative breast cancer.

Illustrative embodiment 25. A method of treating cancer or reducing the occurrence or severity of metastasis through improving the trafficking of immune cells to a solid cancer during Adoptive Cell Therapy (ACT), the method comprising the step of: administering the composition of any one of illustrative embodiments 1-4d or the system of any one of illustrative embodiments 5-8d to a subject having the solid cancer.

Illustrative embodiment 26. The method of illustrative embodiment 25, wherein the ACT includes: 1) identification of a cancer patient in need of ACT; 2) isolating immune cells from the patient's blood or cancer tumor; 3) expanding the immune cells ex vivo; 4) contacting the expanded immune cells with a fucosyltransferase and fucose or GDP-fucose and; 5) returning the fucosylated immune cells to the patient intravenously.

Illustrative embodiment 27. The method of illustrative embodiment 25 or 26, wherein the ACT utilizes a tumor-infiltrating lymphocyte (TIL)-based therapy requiring a cell surface modification using a fucosyltransferase and fucose or GDP fucose, or products genetically engineered or otherwise, that add fucose to cells and upregulate selectin ligands on the cells.

Illustrative embodiment 28. The method of any one of illustrative embodiments 25-27, wherein the ACT utilizes a chimeric antigen receptor gene-transduced T-cell (CAR-T) based therapy enhanced by cell surface modification using a fucosyltransferase and fucose or GDP-fucose or products genetically engineered or otherwise, that add fucose to cells and upregulate selectin ligands on the cells.

Illustrative embodiment 29. The method of any one of illustrative embodiments 25-28, wherein the ACT utilizes a T-cell receptor gene-transduced T cell (TCR-T) based therapy enhanced by cell surface modification using a fucosyltransferase and fucose or GDP-fucose, or products genetically engineered or otherwise, that add fucose to cells and upregulate selectin ligands on the cells.

Illustrative embodiment 30. The method of any one of illustrative embodiments 25-29, wherein the ACT utilizes a chimeric antigen receptor gene-transduced natural killer (NK) cell (CAR-NK) based therapy enhanced by cell surface modification using a fucosyltransferase and fucose or

GDP-fucose, or products genetically engineered or otherwise, that add fucose to cells and upregulate selectin ligands on the cells.

Illustrative embodiment 31. The method of any one of illustrative embodiments 25-30, wherein the ACT utilizes T cells obtained from the patient, a donor, or an iPSC, which are stimulated ex vivo using tumor cells or products of tumor cells to produce tumor-selective immune cells and which are enhanced by cell surface modification using a fucosyltransferase and fucose or GDP-fucose, or products genetically engineered or otherwise, that add fucose to cells and upregulate selectin ligands on the cells.

Illustrative embodiment 32. The method of any one of illustrative embodiments 9-31, wherein the at least one immune checkpoint inhibitor comprises at least one substance selected from the group consisting of a nucleic acid, a peptide, a protein, an antibody, a peptibody, a diabody, a minibody, a single-chain variable fragment (ScFv), a fragment or variant thereof, and any combinations thereof.

Illustrative embodiment 33. The method of any one of illustrative embodiments 9-32, wherein the at least one immune checkpoint inhibitor comprises at least one substance selected from the group consisting of nivolumab, pembrolizumab, cemiplimab, pidilizumab, dostarlimab, pimivalimab, spartalizumab, camrelizumab, sintilimab, tislelizumab, toripalimab, retifanlimab, and combinations thereof.

Illustrative embodiment 34. The method of any one of illustrative embodiments 9-33, wherein the at least one immune checkpoint inhibitor comprises at least one substance selected from the group consisting of atezolizumab, durvalumab, avelumab, cosibelimab, envafolimab, AUNP12, socazolimab, STI-3031, and combinations thereof.

Illustrative embodiment 35. The method of any one of illustrative embodiments 9-34, wherein the at least one immune checkpoint inhibitor comprises at least one substance selected from the group consisting of ieramilimab, Sym022, TSR-033, Fianlimab, leramilimab, INCAGN2385-101, favezelimab, BI754111, and combinations thereof.

Illustrative embodiment 36. A method for treating cancer in a patient, comprising the step of administering a therapeutically effective amount of the composition of any one of illustrative embodiments 1-4d or the system of any one of illustrative embodiments 5-8d to a patient in need thereof.

Illustrative embodiment 37. A method of enhancing the activity of natural killer (NK), cytotoxic T-cell, or Treg cellular activity in a cancer patient comprising the step of administering a therapeutically effective amount of the composition of any one of illustrative embodiments 1-4d or the system of any one of illustrative embodiments 5-8d.

Illustrative embodiment 38. A method for enhancing antibody-dependent cell-mediated cytotoxicity in a cancer patient comprising the step of administering a therapeutically effective amount of the composition of any one of illustrative embodiments 1-4d or the system of any one of illustrative embodiments 5-8d.

Illustrative embodiment 39. A method of treating diseases, disorders, or alleviating or eliminating the symptoms of diseases and disorders, the method comprising the step of administering to a subject in need of treatment a therapeutically effective amount of a combination of at least one fucosylated immune cell type for adoptive cell therapy (fuco-ACT) and at least one of a PD-1 inhibitor, a PD-L1 inhibitor, and/or a LAG-3 inhibitor, wherein the compounds are administered contemporaneously or within 24 hr of each other.

Illustrative embodiment 40. A method of improving the cancer cell fighting capacity of immune cells by first contacting the immune cells with a fucosyltransferase and UDP Fucose ex vivo such that over 50% of the immune cells so contacted become fucosylated.

Illustrative embodiment 41. The method of illustrative embodiment 40, wherein the immune cells are tumor infiltrating lymphocytes (TILs).

Illustrative embodiment 42. The method of illustrative embodiment 41, wherein the fucosylation of the TILs result in an increased attachment to endothelial cell selectins while in circulation.

Illustrative embodiment 43. The method of illustrative embodiment 41 or 42, wherein the fucosylation of the TILs results in the increased surface expression of the trafficking molecule CD162/PSGL-1 and the chemotactic receptor CD183 (CXCR3), and the stimulatory coregulatory molecule CD137 (41BB).

Illustrative embodiment 44. The method of any one of illustrative embodiments 41-43, wherein the fucosylation of the TILs results in an enhanced cytotoxic T cell (CTL) activity as measured by expression of key components of the cytolytic machinery including at least one of FasL/CD95L, perforin, granzyme, or the ability to kill tumor cells.

Illustrative embodiment 45. The method of any one of illustrative embodiments 41-44, wherein the fucosylation of the TILs exhibit an enhanced frequency of fuco-TIL CTLs forming conjugates with target cells compared with nonfucosylated TIL CTLs.

Illustrative embodiment 46. The method of any one of illustrative embodiments 40-45, wherein the infusion of autologous tumor-derived Fuco-TILs to a patient resulted in significant tumor regression and enhanced tumor control.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. An adoptive cell therapeutic pharmaceutical composition, comprising:
at least one isolated immune cell type that has been fucosylated ex vivo;
at least one immune checkpoint inhibitor; and
at least one pharmaceutically acceptable carrier.

2. The composition of claim 1, wherein the at least one isolated immune cell type is selected from the group consisting of cytotoxic T cells (CTL), regulatory T cells (Treg), helper T cells, NK cells, B cells, dendritic cells, genetically modified versions thereof, and combinations thereof.

3. The composition of claim 1, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPα inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

4. The composition of claim 3, wherein the at least one immune checkpoint inhibitor comprises at least one PD-1 inhibitor and at least one LAG-3 inhibitor.

5. A system, comprising:
a composition comprising at least one immune checkpoint inhibitor (ICI); and
a composition comprising at least one fucosylated immune cell type for adoptive cell therapy (fuco-ACT).

6. The system of claim 5, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPα inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

7. The system of claim 6, wherein the at least one immune checkpoint inhibitor comprises at least one PD-1 inhibitor and at least one LAG-3 inhibitor.

8. The system of claim 6, wherein the at least one immune cell type is selected from the group consisting of cytotoxic T cells (CTL), regulatory T cells (Treg), helper T cells, NK cells, B cells, dendritic cells, genetically modified versions thereof, and combinations thereof.

9. An adoptive cell therapy method, comprising the steps of:
1) identifying a cancer patient in need of adoptive cell therapy;
2) isolating immune cells from the patient's blood or cancer tumor;
3) expanding the immune cells ex vivo;
4) contacting the expanded immune cells with a fucosyltransferase and at least one of fucose or GDP-fucose to provide fucosylated immune cells;
5) returning the fucosylated immune cells to the patient intravenously; and
6) administering at least one immune checkpoint inhibitor to the patient simultaneously or wholly or partially sequentially with the fucosylated immune cells.

10. The method of claim 9, further comprising, after step (4), the step of (4a) separating fucosylated lymphocytes from other components after step (4), and wherein step (5) is further defined as returning the fucosylated lymphocytes to the patient intravenously.

11. The method of claim 10, further comprising, after step (4a), the step of (4b) isolating specific fucosylated lymphocyte types from other fucosylated lymphocytes, and wherein step (5) is further defined as returning the specific fucosylated lymphocyte types to the patient intravenously.

12. The method of claim 9, wherein the fucose transferase (FUT) is selected from the group consisting of FUT1, FUT2, FUT3, FUT4, FUT5, FUT6, FUT7, FUT8, FUT9, FUT10, FUT11, and combinations thereof.

13. The method of claim 9, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPα inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

14. The method of claim 13, wherein the at least one immune checkpoint inhibitor comprises at least one PD-1 inhibitor and at least one LAG-3 inhibitor.

15. The method of claim 9, wherein the immune cells are selected from the group consisting of cytotoxic T cells (CTL), regulatory T cells (Treg), helper T cells, NK cells, B cells, dendritic cells, genetically modified versions thereof, and combinations thereof.

16. A method of treating one or more cancers and reducing the occurrence of metastasis in a cancer patient, the method comprising the step of:
administering to the cancer patient, either simultaneously or wholly or partially sequentially, a combination of at least one ICI and at least one fucosylated immune cell type for adoptive cell therapy (fuco-ACT).

17. The method of claim 16, wherein the at least one immune checkpoint inhibitor is selected from the group consisting of a PD-1 inhibitor, a PD-L1 inhibitor, a LAG-3 inhibitor, a CTLA-4 inhibitor, a TIM-3 inhibitor, a B7-H3 inhibitor, an A2aR inhibitor, a CD73 inhibitor, an NKG2A inhibitor, a PVRIG/PVRL2 inhibitor, a CEACAM1 inhibitor, an FAK inhibitor, a CCL2/CCR2 inhibitor, an LIF inhibitor, a CD47/SIRPα inhibitor, a CSF-1 inhibitor, an IL-1 inhibitor, an IL-8 inhibitor, a SEMA4D inhibitor, an Ang-2 inhibitor, a CLEVER-1 inhibitor, a Phosphatylylserine inhibitor, and combinations thereof.

18. The method of claim 17, wherein the at least one immune checkpoint inhibitor comprises at least one PD-1 inhibitor and at least one LAG-3 inhibitor.

19. The method of claim 16, wherein the at least one immune cell type is selected from the group consisting of cytotoxic T cells (CTL), regulatory T cells (Treg), helper T cells, NK cells, B cells, dendritic cells, genetically modified versions thereof, and combinations thereof.

20. The method of claim 16, wherein the cancer is selected from the group consisting of prostate cancer, skin cancer, ovarian cancer, breast cancer, a non-lymphoid parenchymal organ cancer; a cancer of the head and/or neck; leukemia; a cancer of the retina; a cancer of the esophagus; multiple myeloma; melanoma; colorectal cancer; lung cancer; cervical cancer; endometrium carcinoma; gallbladder cancer; liver cancer; thyroid follicular cancer; gastric cancer; non-small cell lung carcinoma; glioma; urothelial cancer; bladder cancer; prostate cancer; renal cell cancer; infiltrating ductal carcinoma; glioblastoma multiform; and combinations thereof.

21. The method of claim 16, further comprising the step of administering at least one additional treatment to the cancer patient, wherein the at least one additional treatment is selected from the group consisting of radiation, surgery, chemotherapy, immunotherapy, targeted therapy, hormone therapy, stem cell transplant, cryotherapy, laser therapy, precision medicine, and combinations thereof.
